(19) United States
(12) Patent Application Publication
(10) Pub. No.: US 2010/0105770 A1
(43) Pub. Date: Apr. 29, 2010

(54) Therapeutic delivery of carbon monoxide

(76) Inventors: Roberto Angelo Motterlini, Genova (IT); Brian Ernest Mann, Sheffield (GB); David Alistair Scapens, High Peak (GB)

Correspondence Address: WOLF GREENFIELD & SACKS, P.C., 600 ATLANTIC AVENUE, BOSTON, MA, 02210-2206, US

(21) Appl. No.: 12/308,963

(22) PCT Filed: Jul. 4, 2007
(86) PCT No.: PCT/GB2007/002483

§ 371 Date: Dec. 18, 2009

(30) Foreign Application Priority Data
Jul. 5, 2006 (GB) 0613362.3

(51) Int. Cl. A61K 31/28; C07F 13/00; A61P 37/02

(52) U.S. Cl. 514/492; 556/46

(57) ABSTRACT

Compounds, pharmaceutical compositions and methods for the therapeutic delivery of carbon monoxide to humans and other mammals that employ Mn complexes having CO ligands, and additional halogen, monodentate and/or bidentate ligands, wherein the additional ligands do not occupy trans positions relative to each other.

The present invention relates to compounds, pharmaceutical compositions and methods for the therapeutic delivery of carbon monoxide to humans and other mammals. Another use of the compositions and compounds is for organ perfusion. In particular, the invention also relates to methods, compounds and pharmaceutical compositions for carbon monoxide delivery to extracorporeal and isolated organs of humans and other mammals.

Carbon monoxide (CO) is, by common definition, a colourless, odourless, tasteless, non-corrosive gas of about the same density as that of air and is the most commonly encountered and pervasive poison in our environment. Depending on the extent and time of exposure, CO is capable of producing a myriad of debilitating and harmful residual effects to the organism (1). (References (1) to (9) for this prior art section are listed below). The most immediate of these effects, and perhaps the most notorious one, is binding to hemoglobin in the blood stream, which rapidly decreases the oxygen transport capability of the cardiovascular system.

Paradoxically, more than half a century ago it was found that CO is constantly formed in humans in small quantities (2), and that under certain pathophysiological conditions this endogenous production of CO may be considerably increased (3-5). The discovery that hemoglobin, a heme-dependent protein, is required as substrate for the production of CO in vivo (6,7) and the identification of the enzyme heme oxygenase as the crucial pathway for the generation of this gaseous molecule in mammals (8) set the basis for the early investigation of an unexpected and still unrecognized role of CO in the vasculature (9).

A discussion of the studies carried' out in this area are reported in the publication WO 02/092075, which originates from the work of some of the present inventors. The beneficial physiological effects of carbon monoxide (CO) has also been recognized and reported in a number of other publications. As a consequence of these beneficial physiological effects, the literature contains many proposals and studies for providing methods or compounds that have use in delivering therapeutic quantities of carbon monoxide at an appropriate rate to a desired physiological site.

WO 2003/000114 (Beth Israel Deaconess Medical Center) describes a method involving the administration of a carbon monoxide-oxygen (O₂) gaseous mixture to an organ, which helps to prevent organ damage for transplant procedures.

Similarly, WO 03/094932 (Yale University) discloses several methods for the generation of carbon monoxide gas and the subsequent administration of the gas to a patient for the treatment of various disorders.

WO 02/078684 (Sangstat Medical Corporation) discloses methods and pharmaceutical compositions for the treatment of vascular disease and for modulating inflammatory and immune processes by using methylene chloride as a carbon monoxide generating compound.

WO 02/092075 mentioned above and WO 2004/045598, which originate from one or more of the present inventors, discloses metal carbonyls that are carbon monoxide releasing compounds (CORMs) for the therapeutic delivery of CO to an in vivo or an ex vivo physiological target site. Some of the transition metal carbonyl compounds disclosed in these publications are soluble in water, which is desirable for formulating a pharmaceutical composition.

WO 03/066067 (Haas, W. et al) proposes as a class of compounds “CO containing organometallic complexes” for use in the treatment and/or prevention of diseases. Generic examples of organometallic transition metal-carbonyl compounds that fall within this class are described. Amongst these examples, the generic formula for the following organometallic compounds is given:

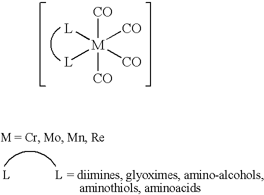

Also listed are compounds of the formula

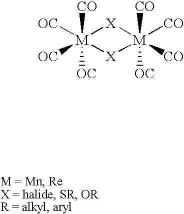

These compounds with Mn—X—Mn bridging are specifically excluded from the present invention.

WO 03/066067 does not describe the synthesis of any of the above compounds and does not contain any literature reference to a procedure for their preparation. It is further noted that there is no evidence in this document, such as biological test data, in support of the use of these compounds for the delivery of CO in vivo or ex vivo.

### STATEMENT OF THE INVENTION

As exemplified by the data presented below, the present inventors have found that pharmaceutical compositions and compounds according to the invention are suitable for use to deliver CO to a physiological target and are able to release CO at relatively high release rates.

Accordingly, a first aspect of the present invention provides a pharmaceutical composition comprising as an active ingredient a compound or ion of the formula (I):

Mn(CO)₄XY (I)

or, when (I) is a compound, a pharmaceutically acceptable salt thereof,

the composition further including, when (I) is an ion, a pharmaceutically acceptable counter-ion,

wherein X and Y do not occupy trans positions in the molecule relative to each other, and

wherein X and Y are the same or different and

each of X and Y is selected from halogens and monodentate ligands bonding to Mn through one of O and S, or X and Y are together a bidentate ligand bonding to MO through O, S or both O and S.

Preferably the compound or ion of the formula (I) has only one Mn atom, i.e. compounds including a Mn—Mn bond or a bridge between two Mn atoms are preferably excluded.

In other embodiments, the compound or ion of the formula (I) has two or more Mn atoms. Preferably, the Mn atoms are connected by a bridge. However, the most preferred compounds or ions of the formula (I) have only one Mn atom.

The species of formula (I) is preferably neutral or an anion, since a cationic form may inhibit release of CO.

Examples of species of formula (I) are:
(a)

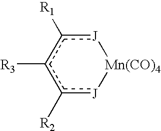

wherein:

each J is independently selected from O or S, preferably both being O,

each of R₁, R₂ and R₃ is independently selected from H (preferably both of R₁ and R₂ not being H and more preferably neither of R₁ and R₂ being H), alkyl or alkenyl of 1 to 6 C atoms (or substituted by halogen, or —OH, —CN or —NH₂, and preferably of 1 to 4 C atoms), or R₂ is as above and R₁ and R₃ taken together, and together with the carbon atoms to which they are attached, are an aromatic ring structure, e.g. phenyl.

An example of this bidentate ligand is [R₁—CO—CH—CO—R₂]⁻ where for example R₁ is —CH₃ and R₂ is —CF₃.
(b) species in which one or both of X and Y are each di-thiocarboxylate bonding through one S atom to Mn or X and Y taken together are di-thiocarboxylate bonding through both S atoms, the di-thiocarboxylate in either case being

[S₂CT]⁻

wherein T is —NR₁R₂ (wherein R₁ and R₂ are selected from H and optionally substituted alkyl (preferably of 1 to 6 C atoms) or R₁ and R₂ are together provided by optionally substituted alkane-di-yl having 1 to 3 C atoms), or —OR wherein R is optionally substituted alkyl' preferably of 1 to 6 C atoms.

Preferred examples are given below.

Other examples are:

R—CO—N—CS₂Mn(CO)₄

where R is alkyl of 1 to 4 C atoms, e.g. methyl or ethyl

C₂H₅—O—CS₂Mn(CO)₄
(c) species in which X and Y together are provided by the bidentate ligand

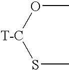

in which S and O bond to Mn and T is optionally substituted alkyl or alkenyl of 1 to 6 C atoms, preferably 1 to 4 C atoms, —NR₁R₂ (wherein R₁ and R₂ are selected from H and optionally substituted alkyl (preferably of 1 to 6 C atoms) or R₁ and R₂ are together provided by optionally substituted alkane-di-yl (preferably having 2 to 6 C atoms), or —OR wherein R is optionally substituted alkyl preferably of 1 to 6 C atoms.

Examples are

(CH₃)₂NCSOMn(CO)₄

RCSOMn(CO)₄ wherein R is preferably alkyl of 1 to 4 C atoms.
(d) species in which a bidentate ligand bonds to Mn, of the formulae

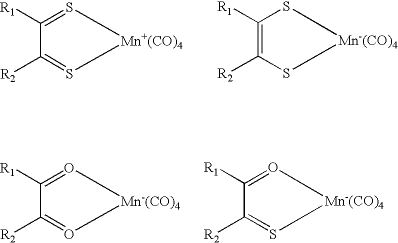

wherein each of R₁ and R₂ is independently —H or optionally substituted alkyl or alkenyl of 1 to 6 C atoms or R₁ and R₂ taken together are an optionally substituted mono- or polynuclear aromatic group.

Examples are

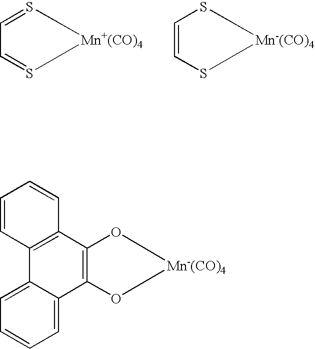

(e) R—SO₂—Mn(CO)₄

in which the two O atoms of —SO₂ bond to Mn, and R is optionally substituted alkyl or alkenyl of 1 to 6 C atoms, preferably 1 to 4 C atoms.

An example is CH₃—SO₂—Mn(CO₄).
(f) (RS)₂Mn⁻(CO)₄

wherein each R is independently selected from optionally substituted alkyl or alkenyl of 1 to 6 C atoms, preferably 1 to 4 C atoms.

In this specification, including the claims, where a group such as alkyl, alkenyl, arylalkyl, arylalkenyl, alkane-di-yl, alkene-di-yl and aromatic group, is specified as “optionally substituted”, the optional substituents are selected from
—COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —N(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,
   wherein R′ is optionally substituted alkyl or alkenyl of 1 to 6 C atoms.

The terms alkyl, alkenyl, alkane-di-yl, alkene-di-yl etc., refer to straight-chain and branched-chain radicals, including cyclic structures where 6 or more C atoms may be present.

Compounds falling, within the definitions (a) to (f) above are believed to be known in themselves in the literature, but not suggested for pharmaceutical use.

Preferably, in the pharmaceutical composition of the invention, (i) each of X and Y is selected from halogen and

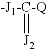

wherein each of J₁ and J₂ is independently selected from O and S and Q is optionally substituted alkyl, alkenyl, aryl, arylalkyl or arylalkenyl, or

(ii) X and Y taken together are a bidentate ligand selected from

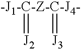

wherein each of J₁, J₂, J₃ and J₄ is independently selected from O and S and Z is optionally substituted alkane-di-yl or alkene-di-yl, or

(iii) X and Y taken together are provided by

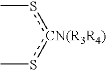

wherein each of R₃ and R₄ is independently selected from H and optionally substituted alkyl, or R₃ and R₄ are together provided by optionally substituted alkane-di-yl or alkene-di-yl having 3 to 6 C atoms or —R₅—O—R₆— wherein each of R₅ and R₆ is optionally substituted alkane-di-yl having 1 to 3 C atoms.

More preferably, Q is alkyl or alkenyl having 1 to 10 C atoms, preferably 1 to 4 C atoms, optionally substituted by one or more of
—COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —N(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,
   wherein R′ is alkyl or alkenyl of 1 to 6 C atoms,

Z is alkane-di-yl or alkene-di-yl of 1 to 10 C atoms (preferably 1 to 5 C atoms) optionally substituted by one or more of
—COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —N(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,
   wherein R′ is alkyl or alkenyl of 1 to 6 C atoms, and

each of R₃ and R₄ (when not H), R₅ and R₆ is optionally substituted by any one of:
—COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —N(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,

wherein R′ is alkyl or alkenyl of 1 to 6 C atoms.

Preferably Q is optionally substituted alkyl having 1 to 4 C atoms, or optionally substituted phenyl. More preferably Q is alkyl having 1 to 4 C atoms unsubstituted or substituted by —OH, —OR═, —COOH, —COOR═, —NH₂, —NH—COOH or —NH—COOR′ where R′ is alkyl having 1 to 4 C atoms, or

phenyl.

Preferably Z is CH₂, CH₂CH₂ or CH(CH₃). Preferably R₃ and R₄ are each selected from alkyl having 1 to 4 C atoms unsubstituted or substituted by —OH, —OR′, —COOH, —COOR′, —NH₂, —NH—COOH or —NH—COOR′ where R′ is alkyl having 1 to 4 C atoms.

The invention further consists in the use of the compounds or ions defined above as the active ingredient, in medicine.

In a second aspect, the invention provides a compound having an anion of the formula (II):

Mn(CO)₄XY (II)

and a counter-cation,

wherein X and Y do not occupy trans positions in the molecule relative to each other, and

wherein X and Y are the same or different and

(i) each of X and Y is selected from

—O—CO-Q

wherein Q is optionally substituted alkyl, alkenyl, aryl, arylalkyl or arylalkenyl, or

(ii) X and Y taken together are a bidentate ligand selected from

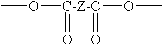

wherein Z is optionally substituted alkane-di-yl or alkene-di-yl.

In this aspect, preferably Q is alkyl or alkenyl having 1 to 10 C atoms, preferably 1 to 4 C atoms, optionally substituted by one or more of
—COOH; —COOR′; —CONH₂; —CONHR′; —CON(H)₂; —COR′; —F, —Cl, —Br, —I; —CN; —NO₂; —OH; —OR′; —SH; —SR′; —O—CO—R′; —NH₂; —NHR′; —N(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,

wherein R′ is alkyl or alkenyl of 1 to 6 C atoms,

Z is alkane-di-yl or alkene-di-yl of 1 to 10 C atoms (preferably 1 to 5 C atoms) optionally substituted by one or more of
—COOH; —COOR′; —CONH₂; —CONHR′; —CON(R′)₂; —COR′; —F; —Cl, —Br, —I; —CN; —NO₂; —OH;. —OR′; —SH; —SR′; —O—CO—R′; —NH₂; —NHR′; —N(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,

wherein R′ is alkyl or alkenyl of 1 to 6 C atoms.

Most preferably Q is unsubstituted C 1 to 4 alkyl, and Z is unsubstituted C 1 to 4 alkane-di-yl.

In a third aspect of the present invention, there is provided a pharmaceutical composition comprising as an active ingredient a compound or ion of the formula (III):

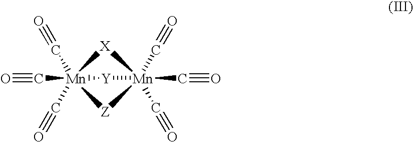

or, when (III) is a compound, a pharmaceutically acceptable salt thereof,

the composition further including, when (III) is an ion, a pharmaceutically acceptable counter-ion,

wherein each X, Y and Z is a halogen or a mondentate ligand bonding through O or S, or a bidentate ligand bonding through O, S or both O and S,

wherein X, Y and Z are the same or different, and

wherein X, Y and Z do not occupy trans positions relative to each other about either of the two Mn atoms.

Preferably the species of formula (III) is neutral or an anion, since a cationic form may inhibit release of CO.

The compound or ion of formula (III) is shown having three bridging ligands. According to a classical electron-counting analysis of the compound or ion structure, there is no Mn—Mn metal bond. However, the distance between the Mn atoms—as obtained from the X-ray crystal analysis of compounds and ions for use in the present invention—does not preclude the existence of some form of bonding interaction between these Mn atoms.

Where X, Y or Z is a monodentate ligand, the ligand may be selected from the preferred ligands described in relation to the monodentate ligands X and Y in the compound or ion of formula (I).

Where X, Y or Z is a halogen, the halogen is preferably Cl, Br or I. Most preferably, the halogen is Cl.

Preferably, in the pharmaceutical composition of the invention (III), each of X, Y and Z is a ligand selected from

(i)

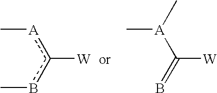

and A and B are independently selected from O and S, and W is optionally substituted alkyl, alkenyl, aryl, arylalkyl, arylalkenyl or W is the group —N(R₃R₄), wherein each of R₃ and R₄ is independently selected from H and optionally substituted alkyl, or R₃ and R₄ are together provided by optionally substituted alkane-di-yl or alkene-di-yl having 3 to 6 C atoms or —R₅—O—R₆— wherein each of R₅ and R₆ is optionally substituted alkane-di-yl having 1 to 3 C atoms;

(ii)

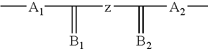

wherein each of A₁, A₂, B₁ and B₂ is independently selected from O and S, and Z is optionally substituted alkane-di-yl or alkene-di-yl; or

(iii)

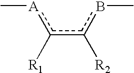

wherein A and B are independently selected from O and S, and each of R₁ and R₂ is independently hydrogen or optionally substituted alkyl or alkenyl of 1 to 6 C atoms, or R₁ and R₂ taken together are an optionally substituted mono- or polynuclear aromatic group.

More preferably, W is alkyl or alkenyl having 1 to 10 C atoms, preferably 1 to 4 C atoms, optionally substituted by one or more of
—COOH, —CSOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —N(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,

wherein R′ is alkyl or alkenyl of 1 to 6 C atoms,

Z is alkane-di-yl or alkene-di-yl of 2 to 10 C atoms (preferably 1 to 5 C atoms) optionally substituted by one or more of
—COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —NH(R′)₂; —NH—CO—R′; —NR′—CO—R′; —NR′—SO₂H, —NH—SO₂H; —NR′—SO₂R′, —NR′—SO₂R′; —OSO₂R′; —C₅₋₂₀aryl; —C₁₋₇alkyl-C₅₋₂₀aryl; —C₁₋₇alkenyl-C₅₋₂₀aryl,

wherein R′ is alkyl or alkenyl of 1 to 6 C atoms, and

each of R₃ and R₄ (when not H), R₅ and R₆ is optionally substituted by any one of:
—COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —NH(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,

wherein R′ is alkyl or alkenyl of 1 to 6 C atoms.

Preferably A and B are the same, A₁ and B₁ are the same, or A₂ and B₂ are the same. A₁, B₁, A₂ and B₂ may all be the same. Alternatively, A₁ and A₂ are the same, or B₁ and B2 are the same.

Preferably, each of X, Y or Z is:

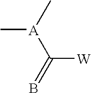

where A, B and W are as defined above.

Most preferably, each X, Y and Z is a halogen, acetyl or thioacetyl ligand.

W may be optionally substituted alkyl having 1 to 4 C atoms, or W may be optionally substituted phenyl. Most preferably W is alkyl having 1 to 4 C atoms unsubstituted or substituted by —OH, —OR′, —COOH, —COOR′, —NH₂, —NH—COOH or —NH—COOR′ where R′ is alkyl having 1 to 4 C atoms, or W is phenyl. W may be unsubstituted alkyl having 1 to 4 C atoms,

Z may be unsubstituted C 1 to 4 alkane-di-yl. Preferably, Z is CH₂, CH₂CH₂ or CH(CH₃).

An example ion according to the third aspect of the invention is: [(OC)₃Mn(μ-OCOCH₃)₃Mn(CO)₃]⁻. This ion may also be represented thus:

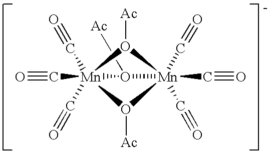

Preferred ions for use in the composition of the third aspect of the invention^{,} include [Mn₂(CO)₆(Boc-Alanine)₃[⁻ and [Mn₂(CO)₆Cl_{3]}⁻ in addition to the ion given above.

In a fourth aspect of the invention there is provided a compound or ion having the formula (IV)

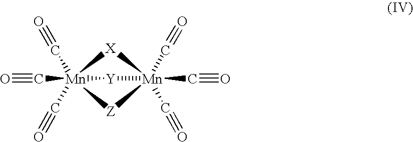

wherein each X, Y and Z is a mondentate ligand bonding through O or S, or a bidentate ligand bonding through O, S or both O and S,

wherein X, Y and Z are the same or different, and

wherein X, Y and Z do not occupy trans positions relative to each other about either of the two Mn atoms.

Where the fourth aspect provides an ion, it will be understood that there is an overall positive or negative charge associated with the structure of formula (IV). The charge may be a −1, −2 or −3 charge, or a +1, +2 or +3 charge.

The preferences for the monodentate and bidentate ligands of the compounds or ions in the compositions of the third aspect of the invention also apply to the ligands of the anions of the fourth aspect of the invention. Preferably, where the fourth aspect provides an ion, the ion has a pharmaceutically acceptable counter-ion.

The pharmaceutical compositions of the present invention typically comprise a pharmaceutically acceptable excipient, carrier, buffer, stabiliser or other materials well known to those skilled in the art.

Such materials should be non-toxic and should not interfere unduly with the efficacy of the active ingredient. The precise nature of the carrier or other material may depend on the route of administration, e. g. oral, intravenous, transdermal, subcutaneous, nasal, inhalatory,, intramuscular, intraperitoneal, or suppository routes.

Pharmaceutical compositions for oral administration may be in tablet, capsule, powder or liquid form. A tablet may include a solid carrier such as gelatin or an adjuvant or a slow-release polymer. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Pharmaceutically, acceptable amounts of other solvents may also be included, in particular where they are required for dissolving the particular metal carbonyl compound contained in the composition.

For intravenous, cutaneous or subcutaneous injection, or injection at the site of affliction, the active ingredient will typically be in the form of a parenterally acceptable solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. Delivery systems for needle-free injection are also known, and compositions for use with such systems may be prepared accordingly.

Administration is preferably in a prophylactically effective amount or a therapeutically effective amount (as the case may be, although prophylaxis may be considered therapy), this being sufficient to show benefit to the individual. The actual amount administered, and rate and time-course of administration, will depend on the nature and severity of what is being treated. Prescription of treatment, e. g. decisions on dosage etc, is within the responsibility of general practitioners and other medical doctors, and typically takes account of the disorder to be treated, the condition of the individual patient, the site of delivery, the method of administration and other factors known to practitioners.

Examples of the techniques and protocols mentioned above can be found in Remington's Pharmaceutical Sciences, 16th edition, Osol, A. (ed), 1980.

When formulating pharmaceutical compositions according to the present invention, the toxicity of the active ingredient and/or the solvent must be considered.

The balance between medical benefit and toxicity should be taken into account. The dosages and formulations of the compositions will typically be determined so that the medical benefit provided outweighs any risks due to the toxicity of the constituents.

A fifth aspect of the invention is a method of introducing, CO to a mammal comprising the step of administering a pharmaceutical composition or compound according to the present invention as defined above. The method of introducing, CO is preferably for treatment of hypertension, such as acute, pulmonary and chronic hypertension, radiation damage, endotoxic shock, inflammation, inflammatory-related diseases such as asthma and rheumatoid arthritis, hyperoxia-induced injury, apoptosis, cancer, transplant rejection, arteriosclerosis, post-ischemic organ damage, myocardial infarction, angina, haemorrhagic shock, sepsis, penile erectile dysfunction and adult respiratory distress syndrome.

The data presented herein is an extension of the work presented in WO 02/092075 and WO 2004/045598. Based on the work presented in these documents, it is preferred that the method of the present invention is for the treatment of hypertension, such as acute, pulmonary and chronic hypertension, endotoxic shock, inflammation, inflammatory-related diseases such as asthma and rheumatoid arthritis, hyperoxia-induced injury, cancer, transplant rejection, arteriosclerosis, post-ischemic organ damage, myocardial infarction, angina, haemorrhagic shock, sepsis and adult respiratory distress syndrome. More preferred is a method for the treatment of hypertension, endotoxic shock, inflammation, inflammatory-related diseases such as asthma and rheumatoid arthritis, post-ischemic organ damage, myocardial infarction and sepsis. Even more preferred is a method for the treatment of hypertension, post-ischemic organ damage and myocardial infarction.

The present aspect of the invention also includes a method of treatment of an extracorporeal or isolated organ, comprising contacting the organ with a pharmaceutical composition according to the present invention. The metal carbonyl makes available carbon monoxide (CO) to limit post-ischemic damage. The organ treated in the method of the invention is an organ which is isolated from the blood supply. The organ may be extracorporeal e.g. a donated organ outside the donor's body and outside the recipient's body, or it may be isolated in the sense that it is in a patient's body and isolated from the blood supply for surgical purposes.

The organ may be, for example, a circulatory organ, respiratory organ, urinary organ, digestive organ, reproductive organ, neurological organ, muscle or skin flap or an artificial organ containing viable cells.

Most preferably, the organ is a heart, lung, kidney or liver. The contacting with the compositions containing metal carbonyl can be achieved by any method that exposes the organ to the composition e. g. bathing or pumping. Preferably, an isolated organ which is attached to the body, i.e. a bypassed organ, is perfused with the composition. An organ which is extracorporeal is preferably bathed in the composition.

In WO 02/092075 and WO 2004/045598 some of the present inventors demonstrated that metal carbonyl compounds can be used in the treatment of particular diseases. Thus, by extension, the present invention also provides the use of a metal carbonyl compound as herein described in the manufacture of a medicament for delivering CO to a physiological target, particularly a mammal, to provide a physiological effect, e.g. for stimulating neurotransmission or vasodilation, or for treatment of any of hypertension, such as acute, pulmonary and chronic hypertension, radiation damage, endotoxic shock, inflammation, inflammatory-related diseases such as asthma and rheumatoid arthritis, hyperoxia-induced injury, apoptosis, cancer, transplant rejection, arteriosclerosis, post-ischemic organ damage, myocardial infarction, angina, haemorrhagic shock, sepsis, penile erectile dysfunction and adult respiratory distress syndrome. Such medicaments may be adapted for administration by an oral, intravenous, subcutaneous, nasal, inhalatory, intramuscular, intraperitoneal or suppository route. Preferably the present invention excludes delivery of a metal carbonyl or a decomposition product thereof to an organism through the skin or mucosa.

More preferably, the use of a metal carbonyl compound as described herein is in the manufacture of a medicament for the treatment of hypertension, such as acute, pulmonary and chronic hypertension, endotoxic shock, inflammation, inflammatory-related diseases such as asthma and rheumatoid arthritis, hyperoxia-induced injury, cancer, transplant rejection, arteriosclerosis, post-ischemic organ damage, myocardial infarction, angina, haemorrhagic shock, sepsis and adult respiratory distress syndrome. More preferred is a medicament for the treatment of hypertension, endotoxic shock, inflammation, inflammatory-related diseases such as asthma and rheumatoid arthritis, post-ischemic organ damage, myocardial infarction and sepsis. Even more preferred is a medicament for the treatment of hypertension, post-ischemic organ damage and myocardial infarction.

The invention further provides use of the metal carbonyls here described in treatment, e.g. by, perfusion, of a viable mammalian organ extracorporeally, e.g. during storage and/or transport of an organ for transplant surgery. For this purpose, the metal carbonyl is in dissolved form, preferably in an aqueous solution. The viable organ may be any tissue containing living cells; such as a heart, a kidney, a liver, a skin or muscle flap, etc.

A sixth aspect of the invention is a kit for producing a pharmaceutical solution. The kit comprises a compound as described herein and a pharmaceutically acceptable solvent. Some of the compounds described herein release CO upon dissolution. Storage of such CORMs in solution is thus impractical because the CORM will decompose or deactivate and will be unable to deliver CO to the physiological target. It is preferred that such CORMs are prepared using the kit according to the present invention immediately before administration to a human or mammalian patient.

### DEFINITIONS

The term “physiological fluid”, as used herein, pertains to fluid suitable for pharmaceutical administration to a physiological system, such as water or a saline solution, or to a fluid already present in a physiological system, such as blood plasma or blood.

### Counter-Ions

Any suitable counter-ions may be employed, bearing in mind for example toxicity. Examples of cations are Na⁺ and K⁺ and ammonium and substituted ammonium ions. Preferably in a quaternary ammonium ion, no H is attached to N, e.g. as in [Me₄N]⁺ and [Me₃NCH₂CH₂OH]⁺. See also the Berge and Stahl references in the next paragraph below.

Examples of counter ions for use in the present invention also include [(15-crown-5)Na]⁺. Species of formula (I) and (III) may also be prepared with a counter ion such as [Ph₃PNPPh₃]⁺. As noted above,, the counter ion in the compositions of the invention is a pharmaceutically acceptable Counter ion, therefore [Ph₃PNPPh₃]⁺ containing compounds or ions are not deemed suitable for use in the compositions of the present invention. [Me₄N]⁺, K⁺ and [choline]⁺ are the preferred counter ions. [Me₄N]⁺ are K⁺ most preferred.

### Salts

It may be convenient or desirable to prepare, purify, and/or handle a corresponding salt of the active compound, for example, a pharmaceutically acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic, such as an acidic group (e.g., —COOH may be —COO⁻; —CSOH may be —CSO⁻ or COS⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NR₄⁺).

Unless otherwise specified, a reference to a particular compound also include salt forms thereof.

### Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate.

Unless otherwise specified, a reference to a particular compound also include solvate forms thereof.

### Ligand Co-Ordination

The compounds and ions according to the first and second aspects of the invention are limited to compounds and ions having ligands X and Y that do not occupy trans (or opposed) positions in the molecule relative to each other. It will be apparent that the ligands X and Y occupy cis positions relative to each other.

An octahedral Mn compound or ion having ligands X and Y that do not occupy trans positions in the molecule relative to each other may be illustrated thus:

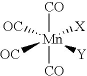

The compounds and ions of according to the third and fourth aspects of the invention are limited to compounds having ligands X, Y and Z that do not occupy trans positions relative to each other about each Mn atom. It will be apparent that the ligands X, Y and Z occupy cis positions relative to each other.

An Mn atom within a compound with two Mn atoms connected by bridging ligands X, Y and Z that do not occupy trans positions relative to each other about each Mn atom may be illustrated thus:

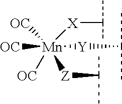

It will be appreciated that the dashed lines on ligands X, Y and Z indicate that these ligands are each bound to a second Mn atom. The second atom has the same co-ordination of ligands to that of the first Mn atom.

Throughout this application references to medical treatment are intended to include both human and veterinary treatment, and references to pharmaceutical compositions are accordingly intended to encompass compositions for use in human or veterinary treatment.

### BRIEF DESCRIPTION OF THE DRAWINGS

Experimental data illustrating the present invention will now be described by reference to the accompanying figures, in which:

FIGS. 1a to 1k are a table presenting solubility information, CO release data, CO stretching frequency, cytotoxicity data and anti-inflammatory data for some of the compounds according to the present invention and some comparative compounds.

FIG. 2 shows the release of CO over time from CORM-349, CORM-371 and CORM-376 measured with the CO electrode.

FIG. 3 shows the degree of contraction over time of pre-contracted rat aorta treated with varying concentrations of (a) CORM-371; (b) CORM-376 and (c) CORM-376 with the guanylate cyclase inhibitor ODQ and glibenclimide (Gli).

### EMBODIMENTS OF THE INVENTION AND EXPERIMENTAL DATA

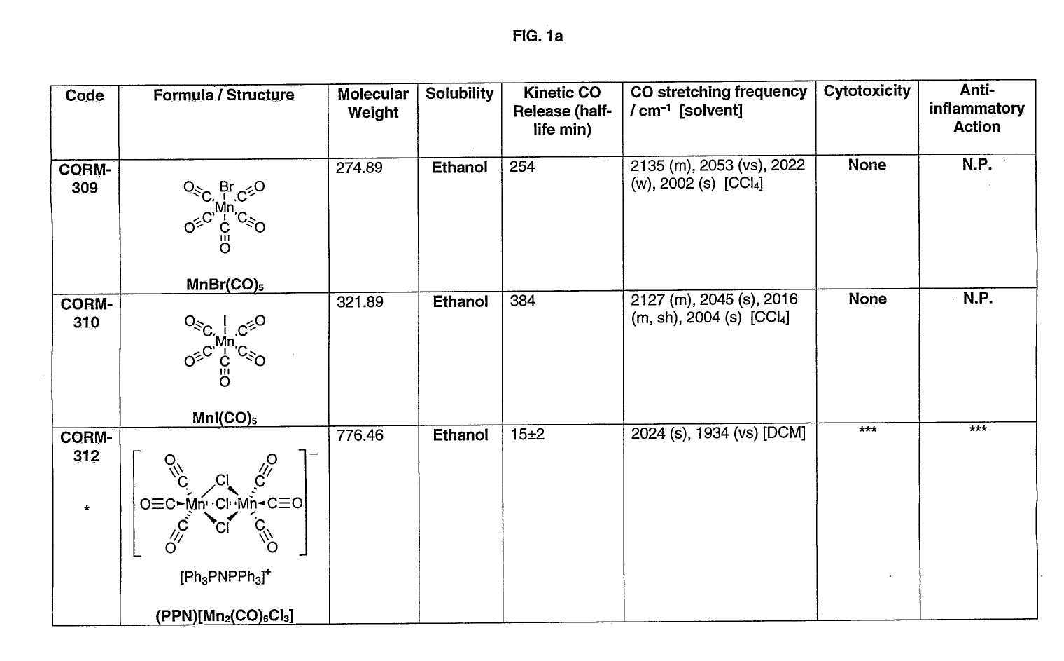

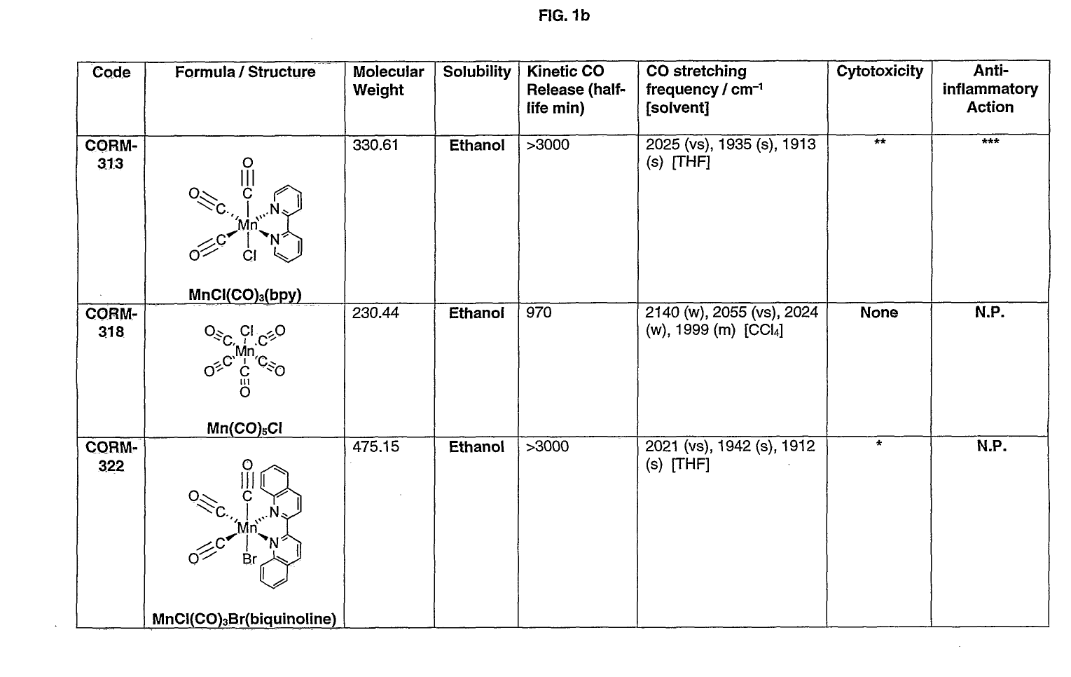

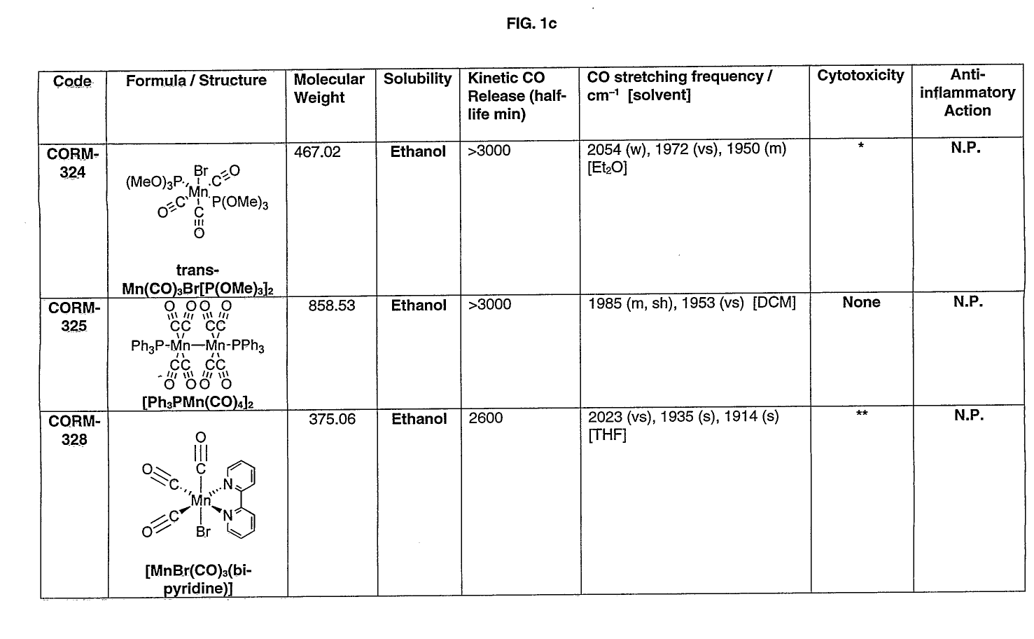

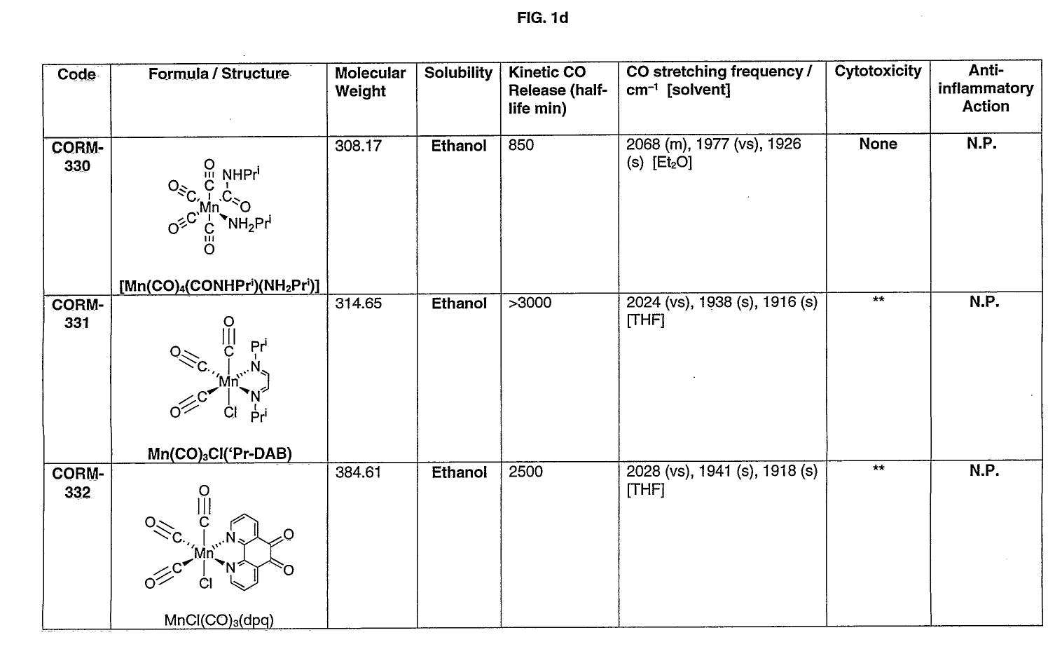

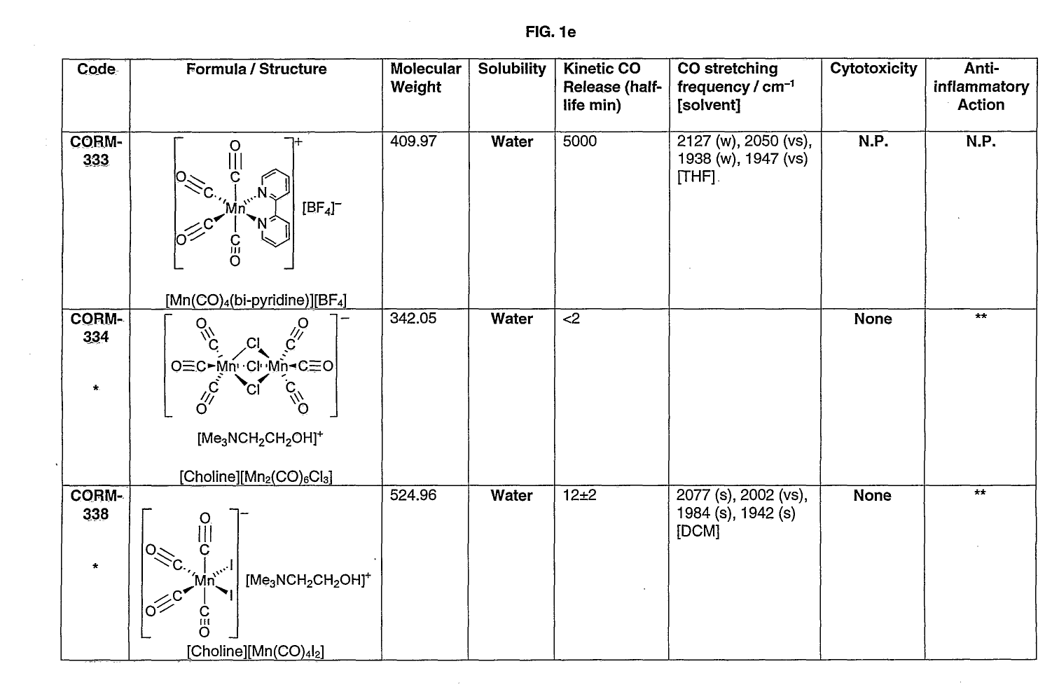

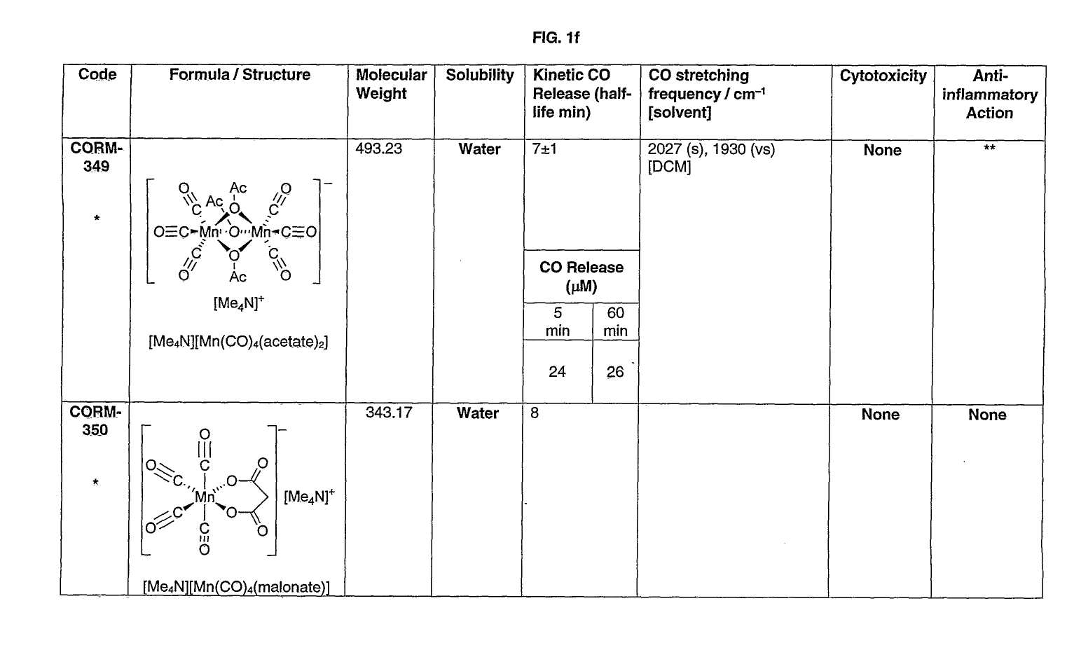

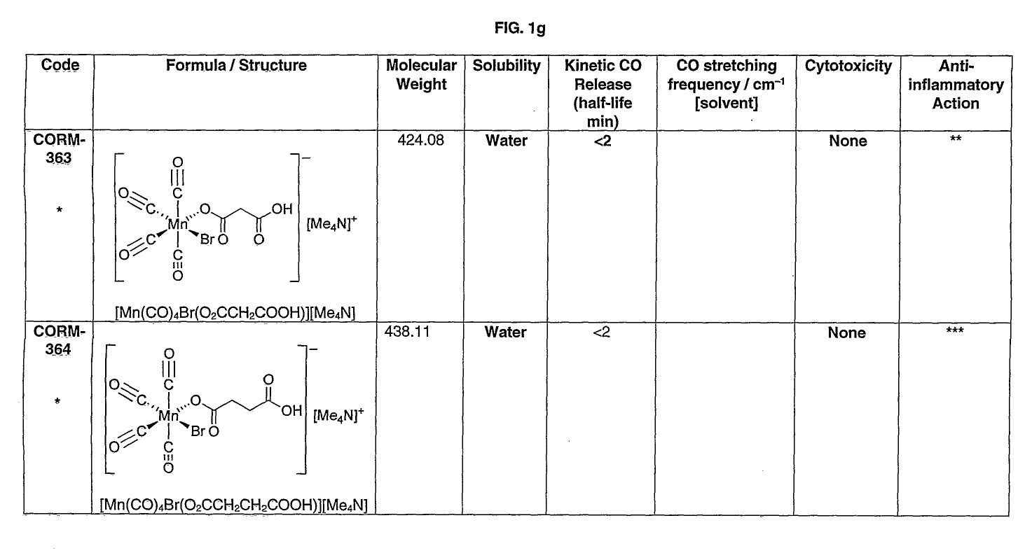

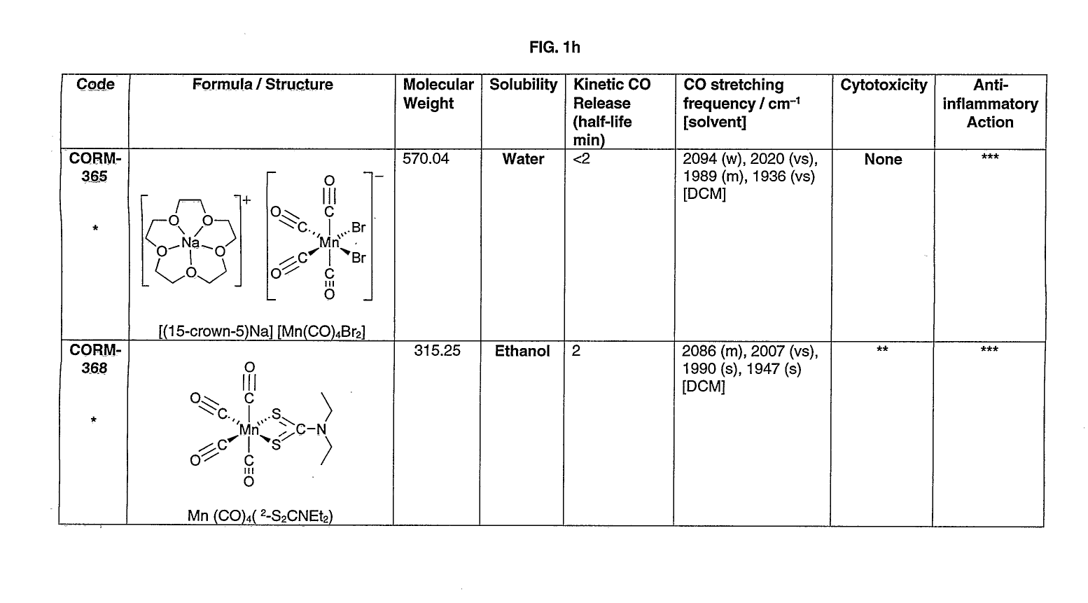

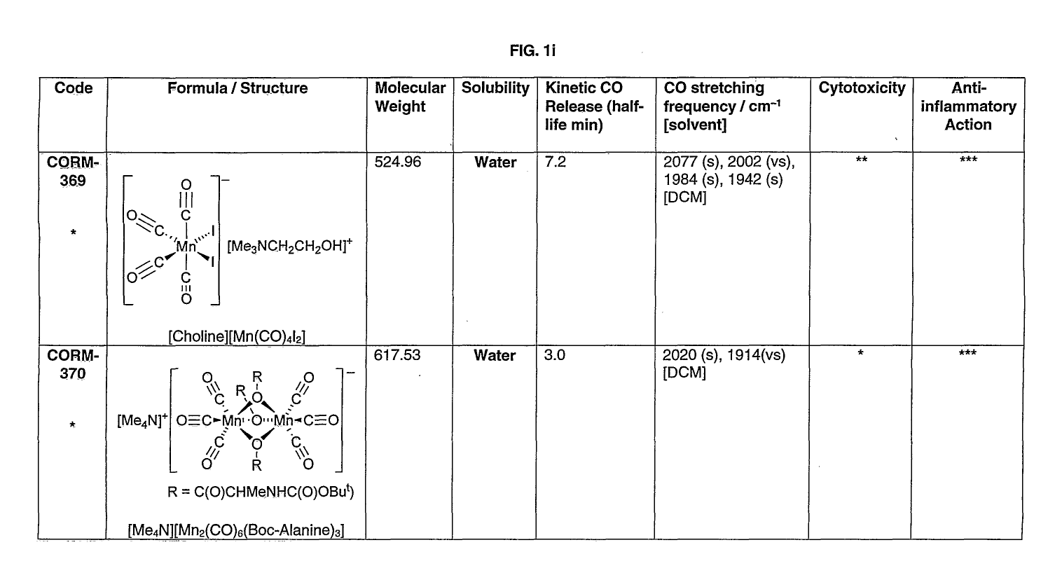

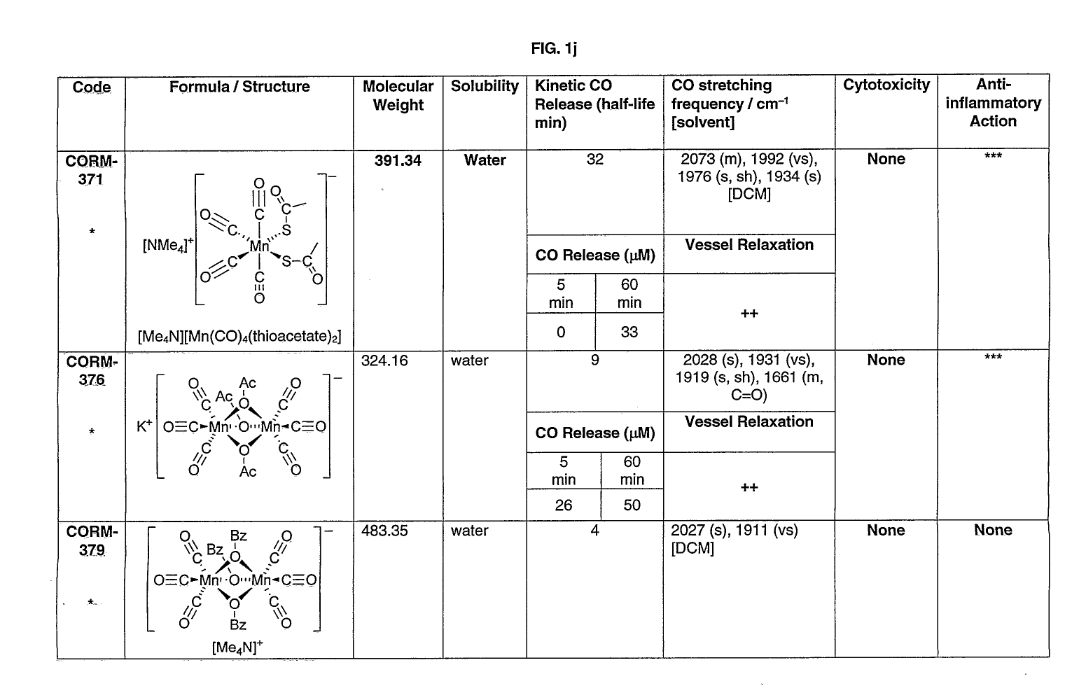

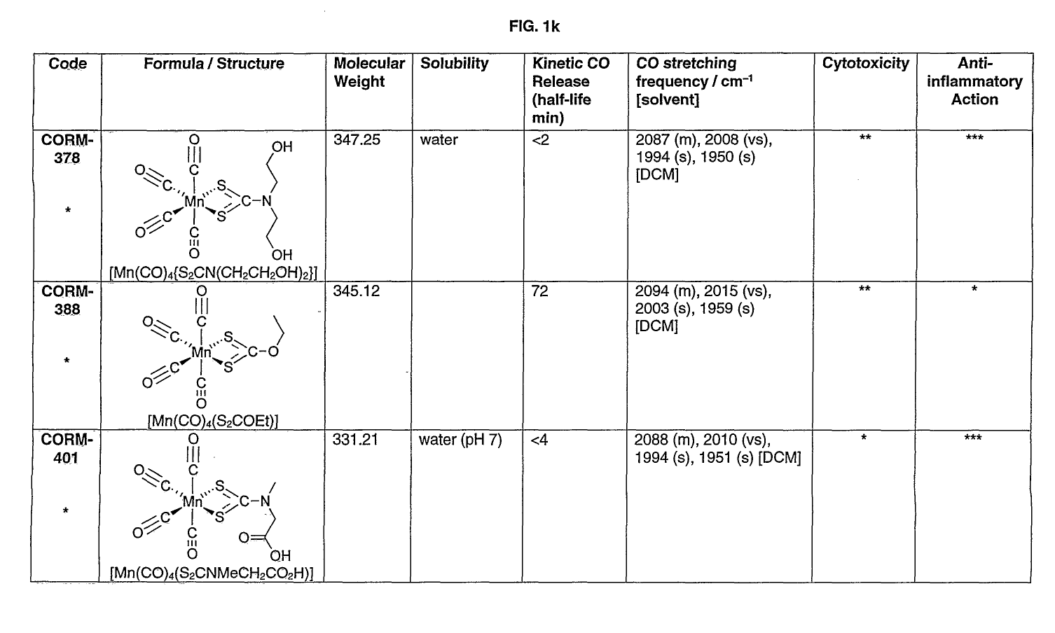

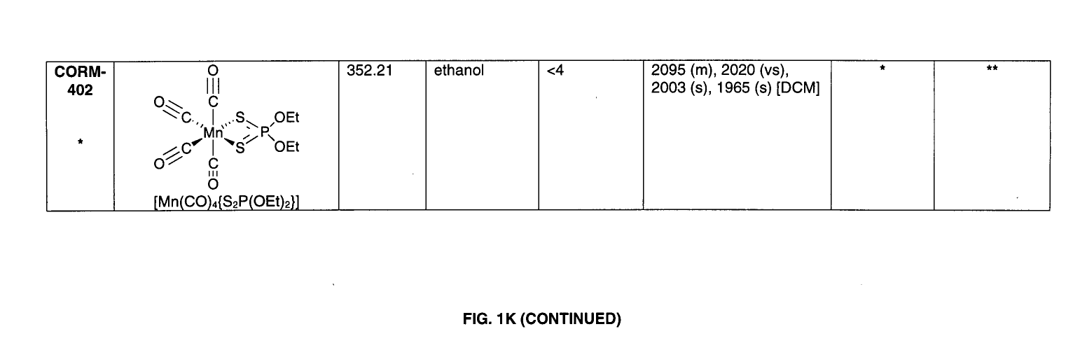

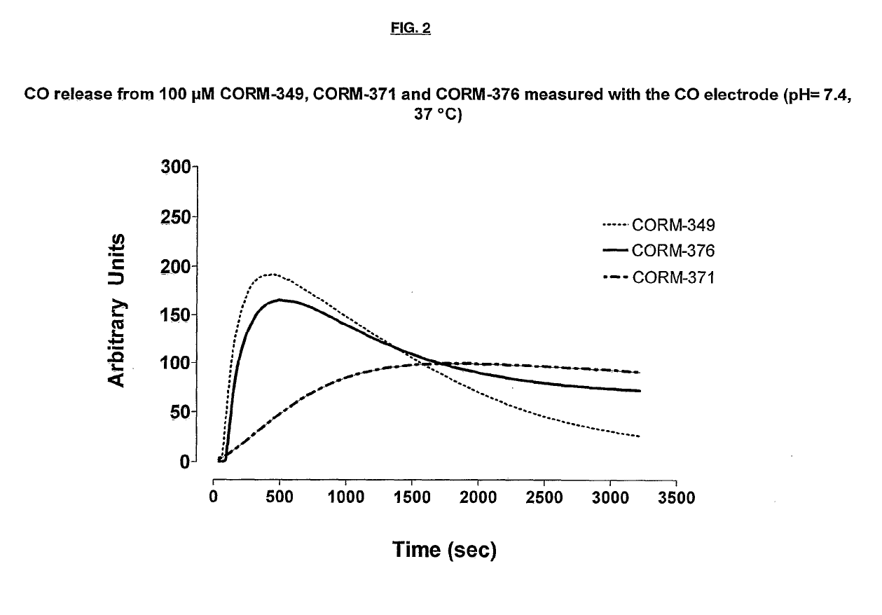

In FIGS. 1a-1k the first column gives the identifying numbers used internally by the applicants.

The data recorded in the figures is explained below:

(1) Cytotoxicity was measured in RAW264.7 macrophages incubated for 24 h with 10, 50 or 100 μM of each compound. The loss in cell viability was measured as a percentage of control. * indicates toxicity detected at 100 μM; ** indicates toxicity detected at 50 μM; *** indicates toxicity detected at 10 μM; “None” indicates that cells were viable and no toxicity was detected up to 100 μM; N.P. indicates assay not performed.

(2) The anti-inflammatory action was measured in RAW264.7 macrophages incubated for 24 h with 10, 50 or 100 μM of each compound in the presence or absence of Lipopolysaccharide (LPS) (1 μg/ml). Nitrite was used as an indicator of inflammation. * indicates a reduction in inflammation detected at 100 μM; ** indicates a reduction in inflammation detected at 50 μM; *** indicates a reduction in inflammation detected at 10 μM; “None” indicates there was no effect of the compound on inflammation; N.P. indicates assay not performed.

(3) The experiments using the isolated aortic rings were conducted to assess the extent of vasorelaxation. One hundred micromolar (100 μM) of each compound were added to a pre-contracted ring and vasorelaxation was assessed as a percentage of the initial contraction, which was expressed as good (+) or very good (++). The sign − indicates that no relaxation was detected.

The release of CO from metal carbonyl complexes was assessed spectrophotometrically by measuring the conversion of deoxymyoglobin (deoxy-Mb) to carbonmonoxymyoglobin (MbCO). MbCO has a distinctive absorption spectrum between 500 and 600 nm, and changes at 540 nm were used to quantify the amount of CO liberated. Myoglobin solutions were prepared freshly by dissolving a known concentration of the protein in phosphate buffer, which was also made up to a known concentration and pH. Sodium dithionite (0.1%) was added to convert myoglobin to deoxy-Mb prior to each reading. The CORM was dissolved in the solvent specified in the solubility column of the table of FIGS. 1a-1k, before addition to the myoglobin solution.

The release of CO from metal carbonyl complexes was also detected using a prototype electrode purchased from World Precision Instrument (Stevenage, Herts, UK). The CO electrode is a membrane-covered amperometric sensor which has been designed on a basic operating principle similar to the nitric oxide (NO) sensor. In fact, the CO sensor can be connected to the ISO-NO Mark II meter for detection of the current signals providing that the poise potential is set to a different value (900 mV for CO as opposed to 860 mV for NO). In principle, CO diffuses through the gas permeable membrane and is then oxidized to CO₂ on the working electrode. This oxidation will create a current whose magnitude can be related directly to the concentration of CO in solution. The CO sensor was used to generate standard curves and calculate the rates of CO release from a CORM compound at different pHs and temperatures. The electrode was immersed into the solutions at different pHs and equilibrated for 30 min prior to addition of the CORM compound. The experiments were maintained at the desired temperature using a Grant W6 thermostat (Cambridge). This method is described in the applicants' earlier publication WO 2005/114161.

### Cell Culture and Biological Assays

The assays correspond to those described in Sawle et. al., British Journal of Pharmacology (2005) 145, 800-810, to which reference should be made.

Murine RAW264.7 monocyte macrophages were purchased from the European Collection of Cell Cultures (Salisbury, Wiltshire, UK) and cultured in Dulbecco's modified Eagle's medium (DMEM) supplemented' with 10% fetal bovine serum, 2 mM L-glutamine, 100 units ml⁻¹ penicillin and 0.1 mg ml⁻¹ streptomycin. Cultures were maintained at 37° C. in a 5% CO₂ humidified atmosphere and experiments were conducted on cells at approximately 80-90% confluence. Macrophages were exposed for 24 hr to LPS (1 μg ml⁻) in the presence or absence of CORMs (10, 50 and 100 μM) and nitrite levels and cytotoxicity were determined at the end of the incubation. Nitrite levels were determined using the Griess method as previously described (Foresti et al. J. Biol. Chem. 272, 18411-18417, (1997)). The measurement of this parameter is widely accepted as indicative of NO production and inflammation. Briefly, the medium from treated cells cultured in 24 well plates was removed and placed into a 96 well plate (50 μl per well). The Griess reagent was added to each well to begin the reaction, the plate was shaken for 10 min and the absorbance read at 550 nm on a Molecular Devices VERSAmax plate reader. The nitrite level in each sample was calculated from a standard curve generated with sodium nitrite (0 μM to 300 μM in cell culture medium). Cell viability was determined using an Alamar Blue assay kit and carried out according to the manufacturer's instructions (Serotec, UK) as previously reported (Clark et al. Biochem. J. 348, 615-619, (2000)). The assay is based on the detection of metabolic activity of living cells using a redox indicator which changes from an oxidised (blue) form to a reduced (red) form. The intensity of the red colour is proportional to the metabolism of the cells, which is calculated as the difference in absorbance between 570 nm and 600 nm and expressed as a percentage of control.

As mentioned, cytotoxicity was measured in mouse RAW264.7 macrophages incubated for 24 h with 10, 50 or 100 μM of each compound. The loss in cell viability was measured as a percentage of control.

As mentioned, the anti-inflammatory action was measured in mouse RAW264.7 macrophages incubated for 24 h with 10, 50 or 100 μM of each compound in the presence or absence of Lipopolysaccharide (LPS) (1 μg/ml). Nitrite in the culture medium was measured as an indicator of inflammation. While the compounds within the scope of the invention generally exhibited anti-inflammatory effects, CORM 350 and CORM 379 did not do so in the test performed. These two compounds are predicted to have useful effects in the treatments discussed herein, because of their rapid CO release.

CO release rates, expressed as a half-life in minutes, are given in FIGS. 1a-1k. Slow release rates (half-life >200 minutes) are indicated for the comparative compounds, while rapid release rates (half-life <50 minutes) were found for compounds within the invention. For example CORM 309, 310 and 318 having five carbonyl ligands have much longer CO release times than corresponding compounds with four carbonyl ligands and two halogen ligands (CORM 334, 338, 365). Compounds having three carbonyl ligands released CO slowly, as did compounds in which a carbon or a nitrogen atom of the ligand bond to the manganese. Slow release is also found for the compound (CORM 325) having Mn—Mn bonding.

The solubility information shows that generally the ionic compounds in which the Mn—CO complex is an anion, are water-soluble, which can be advantageous in biological use. Uncharged complexes, such as CORM 378, can be made water soluble by the presence of suitable ligands.

The CO stretching frequencies are of interest. Normally a high CO stretching frequency, associated with a weak metal-CO bond, is indicative of easy release of CO, but this does not appear to be the case in the compounds of the invention in FIGS. 1a to 1k.

The compounds of the invention, where tested, mainly showed low or zero cytotoxicity. Even a cytotoxic compound may be suitable for use in medicine, either where its benefit outweighs its toxicity, or when its beneficial effect is obtained in a non-absorbable form e.g. when it is bound to a substrate.

As FIGS. 1a-1k indicate, in the compound of the invention, the two ligands other than the carbonyls do not occupy trans (opposed) Mn-bonding positions relative to each other. X-ray data has shown that in CORM 371 the ligands bond to Mn atom through S, not through O.

Vasodilatation data for CORM 371 and CORM 376 was measured as described previously by the inventors in their earlier publication, WO 2004/045599. This is described in more detail below.

### Preparation of Isolated Rat Aortic Rings and Experimental Protocol

The method for the preparation of isolated aortic rings has been previously described (Sammut et al Br J Pharmacol 125: 1437-1444, 1998; Motterlini et al Circ Res 90: E17-E24, 2002). The thoracic aorta was isolated from Sprague-Dawley rats (350-450 g) and flushed with cold Krebs-Henseleit buffer (4° C., pH 7.4) containing (in mM): 118 NaCl, 4.7 KCl, 12 KH₂PO₄, 1.2 MgSO₄.7H₂O, 22 NaHCO₃, 11 Glucose, 0.03 K⁺EDTA, 2.5 CaCl₂ and supplemented with 10 μM indomethacin. Each aorta was trimmed of adventitial tissue and ring, sections (˜3 mm length) were produced from the mid aortic segment. The rings were then mounted between two stainless steel hooks in 9-mL organ baths containing Krebs-Henseleit buffer which was maintained at 37° C. and continuously gassed with 95% O₂-5% CO₂. One hook was attached to a Grass FT03 isometric force transducer whilst the other was anchored to a sledge for regulation of the resting tension of the aortic ring. The rings were initially equilibrated for 30 min under a resting tension of 2 g which was previously determined to be optimal. Continuous recording of tension was made on a Grass 7D polygraph (Grass Instruments, Quincy, Mass.) in combination with a Biopac MP100 system using AcqKnowledge™ software (Linton Instruments, Norfolk, UK). Before each protocol was carried out, rings were contracted with a standard dose of KCl (100 mM) in order to provide an internal reference and to control for variability in contractile responsiveness between tissues. The relaxation response to CORM-3 (25 μM) in the presence or absence of YC-1 (5 μM final concentration, 30 min pre-incubation) was assessed in aortic rings pre-contracted with phenylephrine (1 μmol/L).

### Results

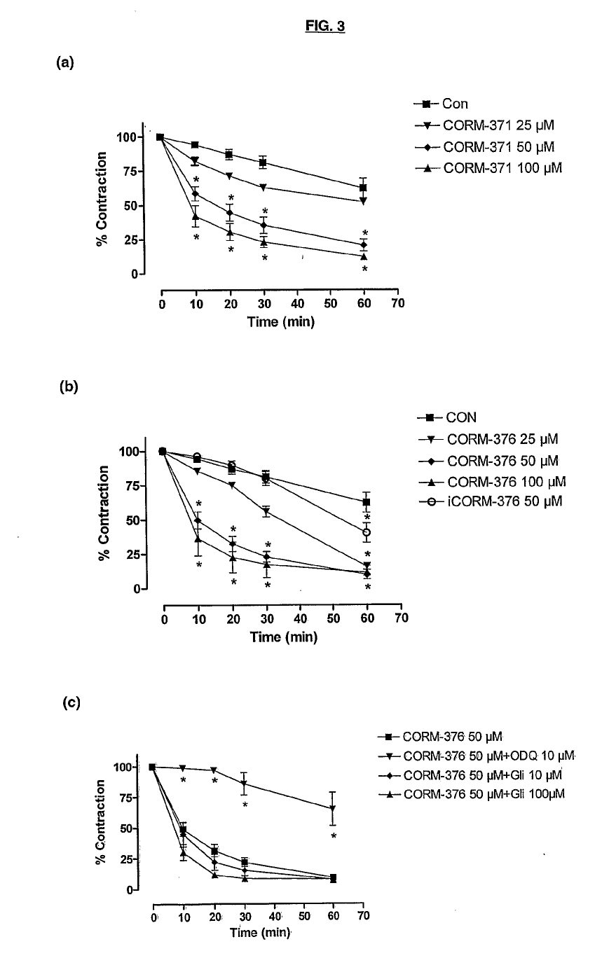

FIG. 3(a) shows that CORM-371 caused a concentration-dependent decrease in contraction following its addition to aortic rings.

FIG. 3(b) shows that CORM-376 caused a concentration-dependent decrease in contraction following its addition to aortic rings. In contrast, contraction remained similar to control when iCORM-376 (the inactive counterpart) was employed in the experiments.

FIG. 3(c) shows that the inhibitor of guanylate cyclase ODQ significantly prevented the vasorelaxation elicited by CORM-376. However, inhibition of ATP-dependent dependent K⁺ channels by glibenclimide at two different concentrations did not affect CORM-376-mediated dilatation. Both CORM-371 and CORM-376 are good vasodilators in the aortic rings model. The mechanisms underlying CORM-376 relaxation appear to involve release of CO and activation of guanylate cyclase to produce cGMP. ATP-dependent K⁺ channels do not seem to participate to CORM-376-mediated dilation processes.

### Syntheses

In this section, the numerals [1], [2], [3] etc. refer to the References listed below. Some of these references relate to compounds having the Mn anion specified and a different cation.

Below M^{r} is the calculated molecular weight. m/z is the molecular weight obtained by mass spectrometry.

### CORM-309 [MnBr(CO)₅] [1]

4.6 g (0.0118 mol) of [Mn₂(CO)₁₀] was dissolved in 50 ml of CCl₄ under nitrogen, and the system stirred for 5-10 min at room temperature. 0.79 ml (0.0152 mol) of Br₂ was then added slowly (5-10 min). The system was then allowed to react at 40° C. for 1 h. Following this, the solvent was removed, and the crude product was washed with 3× portions of water. It was then dried under vacuum.

The crude product was then dissolved in ˜150 ml of CH₂Cl₂, filtered, and then ˜60 ml of hexane was added. The volume of the solution was then reduced slowly on a rotary evaporator to ˜25 ml, by which point the product had precipitated out. It was filtered and washed with several portions of cold petroleum ether (40/60). 4.913 g of an orange solid was obtained. The yield was 76%. M^{r}=274.89.

¹⁷O NMR (CD₂Cl₂): δ(ppm) 383.0 (CO), 388.8 (CO)

⁵⁵Mn NMR (CD₂Cl₂): δ(ppm) −1139 line width 560 Hz

IR (CCl₄) ν(cm⁻¹): 2135 (m), 2053 (vs), 2022 (w), 2002 (s)

Mass Spec (m/z): 274/276 (M⁺), 218/220 (M⁺-2CO), 190/192 (M⁺-3CO), 162/164 (M⁺-4CO), 134/136 (M⁺-5CO)

Elemental: MnC₅O₅Br found (calc) C: 21.73 (21.85), Br: 29.05 (29.07)

### CORM-310 [MnI(CO)₅] [2]

A 1% sodium amalgam was prepared (6 ml Hg and ˜900 mg Na) in a Schlenk tube under nitrogen. To this was added 40 ml of dry THF (tetrahydrofuran) 2.00 g, (5.13 mmol) of [Mn₂(CO)₁₀] was then added and the system stirred vigorously for 40 min.

The now green opaque ‘solution’ was transferred from this first Schlenk tube to a second, which was also under nitrogen. To this was added a solution of I₂ (2.650 g, 10.4 mmol) in 20 ml THF, dropwise (˜30 min). The solution slowly changed to a clear, dark red/brown/orange colour. After complete addition of the 1₂ solution, stirring was continued for a further 10 min.

Solvent was then removed on rotary evaporator. The residue was extracted with 120 ml of a 1:1 CH₂Cl₂/hexane mixture. The extract was then filtered and solvent removed on rotary evaporator.

The product was recrystallised from hexane at −20° C. to give 2.387 g of orange needles. Yield was 72%. M^{r}=321.89.

¹⁷O NMR (CD₂Cl₂): δ(ppm) 385.7 (CO)

⁵⁵Mn NMR (CD₂Cl₂): δ(ppm) −1472 line width 680 Hz

IR (CCl₄) ν (cm⁻¹): 2127 (m), 2045 (s), 2016 (m, sh), 2004 (s)

Mass Spec (m/z): 322 (M⁺), 266 (M⁺-2CO), 238 (M⁺-3CO), 210 (M⁺-4CO), 182 (M⁺-5CO)

Elemental: MnC₅O₅I found (calc) C: 18.64 (18.66), I: 39.68 (39.42)

### CORM-312 [PPN][Mn₂(CO)₆Cl₃] [3]

200 mg (0.87 mmol) of [MnCl(CO)₅] and 350 mg (0.61 mmol) of PPNCl were refluxed together in 10 ml of CH₂Cl₂ for 1 h, under nitrogen. After cooling to room temperature, another 10 ml of CH₂Cl₂ was added to dissolve any product that had come out of solution. The solution was then filtered and 50 ml of hexane was added.

The product precipitated out immediately, although it was allowed to stand for 45 min to ensure complete precipitation. The product was filtered off, washed with hexane and then dried under vacuum. 429 mg of a bright yellow solid was obtained. The yield was 100%.

¹H NMR (CD₂Cl₂): δ(ppm) 7.47 (meta, para, Ph), 7.61 (ortho, Ph)

¹³C NMR (CD₂Cl₂): δ(ppm) 127.0 (ipso, N =108 Hz), 129.6 (meta, N=13 Hz), 132.2 (ortho, N=11 Hz), 133.9 (para), 222.2 (CO)

¹⁷O NMR (CD₂Cl₂): δ(ppm) 383.3 (CO)

⁵⁵Mn NMR (CD₂Cl₂): δ(ppm) −267 line width 3280 Hz

Mass Spec (ES⁻) (m/z): 299 ([Mn₂(CO)₃³⁵Cl_{3]}⁻); 243 ([Mn₂(CO)³⁵Cl₃]⁻])

IR (CH₂Cl₂) ν(cm⁻¹): 2024 (s), 1934 (vs)

Based on the preliminary analytical data, the product was initially identified as [PPN][Mn(CO)₄Cl₂]. However, additional analysis, particularly X ray crystal structure analysis, has revealed that the product has the title structure. The structure [Mn₂(CO)₆Cl₃)⁻ has also been reported. See A. Sieker, A. J. Blake and B. F. G. Johnson, “New mixed carbonyl-nitro and -nitrito complexes of manganese and rhenium,” J. Chem. Soc., Dalton Trans., 1996, 1419-27.

CORM-313 (MnCl(CO)₃(bpy)] [4]

115 mg (0.5 mmol) of [MnCl(CO)₅] (CORM-318) and 78 mg (0.5 mmol) of 2,2′-bipyridine were refluxed together in 15 ml of ether for ˜45 min, under nitrogen. During this time the product precipitated out.

The system was then cooled to −20° C. to ensure complete precipitation and the product collected by filtration. It was washed several times with cold ether and then dried under vacuum. 149 mg of an orange solid was obtained. The yield was 90%. M^{r}=330.61. (m/z) (—Cl) 295. ¹H (δ, ppm), 7.55 (H₅), 8.03 (H₄), 8.17 (H₃), 9.2 (H₆).

¹H NMR (CD₂Cl₂): δ(ppm) 7.55 (t {J=6.1 Hz}, H₅ 1H), 8.03 (t {J=7.4 Hz}, H₄ 1H), 8.17 (d, {J=7.4 Hz), H₃ 1H), 9.2 (d {J=4.9 Hz}, H₆ 1H)

¹³C NMR (CD₂Cl₂): δ(ppm) 122.8 (C₃ or C₅), 126.6 (C₃ or C₅), 138.8 (C₄), 153.6 (C₆), 155.8 (C₂)

¹⁷O NMR (CD₂Cl₂): δ(ppm) 376.9 (CO trans to Cl), 382.7 (COs trans to N)

⁵⁵Mn NMR (CD₂Cl₂): δ(ppm) 174 line width 4950 Hz

IR (THF) ν(cm⁻¹): 2025 (vs), 1935 (s), 1913 (s)

Mass Spec (m/z): 295 (M⁺-Cl), 246/248 (M⁺-3CO), 211 (M⁺-3CO-Cl)

Elemental: MnC₁₃H₈N₂O₃Cl found (calc) C: 46.88 (47.23), H: 2.20 (2.44), N: 8.34 (8.47), Cl: 10.79 (10.72)

### CORM-318 [MnCl(CO)₅]

### Method (a) [1], [2]

2.00 g (5.13 mmol) of [Mn₂(CO)₁₀] was dissolved in the minimum amount of degassed CCl4 (˜40 ml) in an ice bath, under nitrogen. A Cl₂ saturated sample of CCl₄ (12.5 ml) was then added dropwise (˜30 min) with stirring using an equalising pressure dropping funnel. After complete addition, the system was allowed to warm to room temperature and then stirred for a further 4 h.

A yellow precipitate steadily formed. This was filtered off and washed several times with CCl₄ and then dried under vacuum. Mass of product obtained was 0.915 g. The yield was 39%.

¹⁷O NMR (CD₂Cl₂): δ (ppm) 389.2 (CO trans to Cl), 381.5 (CO trans to CO)

⁵⁵Mn NMR (CD₂Cl₂): δ(ppm) −954 line width 340 Hz

IR (CCl₄) ν(cm⁻¹): 2140 (w), 2055 (vs), 2024 (w), 1999 (m)

Mass Spec (m/z): 230/232 (M⁺), 174/176 (M⁺-2CO), 146/148 (M⁺-3CO), 118/120 (M⁺-4CO), 90/92 (M⁺-5CO)

### Method (b) [5]

1.08 g (2.76 mmol) of [Mn₂(CO)₁₀] was dissolved in 75 ml of dry CH₂Cl₂ under nitrogen. 4 ml (0.048 mol) of SO₂Cl₂ was then added fairly slowly (5-10 min). The system was allowed to react for ˜8 days, by which point some of the product had come out of solution and IR showed the reaction to be complete.

The solvent was removed under vacuum and the remaining solid was washed several times with ethanol, and then dried under vacuum. 1.225 g of a yellow solid was obtained. The yield was 96%.

CORM-322 [MnBr(CO)₃(2,2′-biquinolyl)] [6]

137 mg. (0.5 mmol) of [MnBr(CO)5] (CORM-309) and 115 mg (0.45 mmol) of 2,2′-biquinolyl were refluxed together in 10 ml of ether for ˜3 h, under nitrogen. During this time the product precipitated out. Note, an excess of [MnBr(CO)₅] was used because of the insolubility of biquinolyl in ether.

The system was then cooled to −20° C. to ensure complete precipitation and the product collected by filtration. It was washed several times with cold ether and then dried under vacuum. 206 mg of a deep red solid was obtained. The yield was 96%. M^{r}=475.18.

¹H NMR (CD₂Cl₂): δ(ppm) 7.78 (t {J=6.0 Hz} H₆), 8.03 (t {J=6.8 Hz} H₇, H₈), 8.33 (d {J=7.7 Hz}, H₄), 8:55 (d {J=6.6 Hz} H9), 8.99 (d {J=8.8 Hz}, H₃)

⁵⁵Mn NMR (CD₂Cl₂): δ (ppm) 283 line width 3500 Hz

IR (THF) ν(cm⁻¹): 2021 (vs), 1942 (s), 1912 (s)

Mass Spec (m/z): 395 (M⁺-Br), 339 (M⁺-Br-2CO), 311 (M⁺-Br-3CO)

Elemental: MnC₂₁H₁₂N₂O₃Br found (calc) C: 52.85 (53.08), H: 2.36 (2.55), N: 5.84 (5.90), Br: 16.87 (16.82)

### CORM-324 [MnBr(CO)₃{P(OMe)₃}₂] [5]

150 mg (0.542 mmol) of [MnBr(CO)₅] and 105 mg/100 μl (0.848 mmol) of P(OMe)₃ were refluxed together in 8 ml of benzene for 4 h under nitrogen. Following this the solvent was removed to give an orange oil. This was recrystallised from hot petroleum ether (40/60) to give an orange solid.

However, the product was impure, and so it was purified by chromatography. A silica gel column was prepared (40×3 cm) using petroleum ether (40/60). Band 1 (yellow) eluted with 5:1 pet ether/ether. This was identified (by IR) as the mer-trans isomer of [MnBr(CO)₃{P(OMe)₃}₂]. 78 mg of a yellow/brown solid was obtained. The yield was 30.8%.

¹H NMR (CD₂Cl₂): δ(ppm) 3.80 (CH₃, N=11 Hz)

¹³C NMR (CD₂Cl₂): δ(ppm) 53.2 (CH₃), 214.1 (COs trans to P), 218.7 (CO trans to Br)

¹⁷O NMR (CD₂Cl₂): δ(ppm) 373.5 (CO), 61.6 (OMe)

⁵⁵Mn NMR (CD₂Cl₂): δ(ppm): −1267 line width 5100 Hz

IR (Et₂O ) ν(cm⁻¹): 2054 (w), 1972 (vs), 1950 (m)

Mass Spec (m/z): 387 (M⁺-Br), 331 (M⁺-Br-2CO)

Elemental: MnC₉H₁₈P₂O₉Br found (calc) C: 22.80 (23.15), H: 3.45 (3.88), Br: 16.81 (17.11)

Band 2 (yellow) eluted with 3:1 pet ether/ether. This was identified (by IR) as the fac-isomer of [MnBr(CO)₃{P(OMe)₃}₂]. An undeterminable amount of a yellow oil was obtained'. IR (Et₂O) ν(cm⁻¹) 2043 (s), 1977 (s), 1937 (s).

Band 3 (yellow) eluted with 3:1 pet ether/ether. This was identified (by IR) as the tri-substituted product [MnBr(CO)₂{P(OMe)₃}₃] 25 mg of a yellow solid was obtained. The yield was 8.2%. IR (Et₂O) ν(cm⁻¹): 1979 (s), 1900 (s).

### CORM-325 [Mn(CO)₄(PPh₃)]₂ [7]

1.0 g (2.6 mmol) of [Mn₂(CO)₁₀] and 1.33 g (5.2 mmol) of PPh₃ were heated to 130° C. together in 20 ml of pentanol for 2 h, under nitrogen. During this time the solution turned to red and then orange and then the product precipitated. The system was allowed to cool to room temperature and then the product was collected by filtration. It was washed with several portions of petroleum ether (40/60).

The product was recrystallised from benzene/heptane and then washed with heptane and then petroleum ether (40/60). Two crops were obtained (0.511 g and 0.332 g) of an orange solid. Overall yield was 38%. M^{r}=858.54.

¹H NMR (CD₂Cl₂): δ(ppm) 7.45 (mult, meta, para 3H), 7.52 (mult, ortho 2H)

¹³C NMR (CD₂Cl₂): δ(ppm) 137.1 (ipso, ¹J_{CP}=41.1 Hz), 133.0 (ortho, ²J_{CP}=10.6 Hz), 130.3 (para), 128.9 (meta, ³J_{CP}=9.4 Hz), 227.1 (CO)

¹⁷O NMR (CD₂Cl₂): δ(ppm) 380.7 (CO)

³¹P NMR (CD₂Cl₂): δ(ppm) 76.08 (PPh₃)

⁵⁵Mn NMR (CD₂Cl₂): δ(ppm) −2391 line width 187 Hz

IR (CH₂Cl₂) ν(cm⁻¹): 1985 (m, sh), 1953 (vs)

Mass Spec (m/z): 429 (M/21, 401 (M/2+-CO), 317 (M/2+-4CO), (−2PPh₃, −2CO, +H⁺) 279

Elemental: Mn₂C₄₄H₃₀P₂O₈ found (calc) C: 61.89 (61.56), H: 3.66 (3.52)

CORM-328 [MnBr(CO)₃(2,2′-bipyridine)] [7]

275 mg (1 mmol) of [MnBr(CO)5] and 172 mg (1.1 mmol) of 2,2′-bipyridine (i.e. a slight excess) were refluxed together in 20 ml of ether for ˜5 h., under nitrogen. The reaction was monitored by IR spectroscopy until it was evident that there was no more [MnBr(CO)₅] present. During this time the product precipitated out.

The system was then cooled to −20° C. to ensure complete precipitation and the product collected by filtration. It was washed several times with cold ether and then dried under vacuum. 356 mg of an orange solid was obtained. The yield was 95%. M^{r}=375.

¹H NMR (CD₂Cl₂): δ (ppm) 7.78 (t {J=6.0 Hz} H₆), 8.03 (t {J=6.8 Hz} H₇, H₈), 8.33 (d {J=7.7 Hz}, H₄), 8.55 (d {J=6.6 Hz}H₉), 8.99 (d {J=8.8 Hz}, H₃)

¹⁷O NMR (CD₂Cl₂): δ(ppm) Could not be obtained

⁵⁵Mn NMR (CD₂Cl₂): δ(ppm) 283 line width 3500 Hz

IR (THF) ν(cm⁻¹): 2021 (vs), 1942 (s), 1912 (s)

Mass Spec (m/z): 295 (M+-Br), 239 (M⁺-Br-2CO), 211 (M⁺-Br-3CO)

Elemental: MnC₂₁H₁₂N₂O₃Br found (calc) C: 52.85 (53.08), H: 2.36 (2.55), N: 5.84 (5.90), Br: 16.87 (16.82)

Isopropyl-Diazabutadiene (ⁱPr-DAB) [9] Used in CORM-331

7.255 g (0.05 mol) of glyoxal (40% aq. solution) was added to ˜5-10 ml of water, under nitrogen. 10.9 ml (0.128 mol) of isopropylamine was then added dropwise with vigorous stirring, and the reaction became warm. It was stirred for ˜2-3 h.

Following this the product was extracted with 3× portions of ether, dried over magnesium sulphate and then filtered. The resulting solution was taken to dryness on rotary evaporator to give an off-white/light brown solid. This was recrystallised from ether at −80° C. 1.268 g of white needles were obtained. The yield was 18%.

CORM-331 [MnCl(CO)₃(ⁱPr-DAB)] [10]

115 mg (0.5 mmol) of [MnCl(CO)₅] and 70.2 mg (0.5 mmol) of ⁱPr-DAB were refluxed together in 10 ml of ether for ˜1 h, under nitrogen. During this time the product precipitated out.

The system was then cooled to −20° C. to ensure complete precipitation and the product collected by filtration. It was washed several times with cold ether and then dried under vacuum. 140 mg of an orange solid was obtained. The yield was 89%. M^{r}=314.

¹H NMR (CD₂Cl₂): δ (ppm) 1.56 (s, CH₃ 12H), 4.44 (mult, ⁱPr CH 2H), 8.25 (s, imine CH 2H),

¹³H NMR (CD₂Cl₂): δ(ppm) 22.6 (ⁱPr CH₃), 23.0 (ⁱPr CH₃), 64.6 (ⁱPr CH), 159.4 (C═N), 216.4 (CO trans to Cl), 221.8 (CO's trans to N)

¹⁷O NMR (CD₂Cl₂): δ(ppm) 378.4 (CO trans to Cl), 384.6 (COs trans to N)

⁵⁵Mn NMR (CD₂Cl₂): δ(ppm) 131 line width 3100 Hz

IR (THF) ν(cm⁻¹): 2024 (vs), 1938 (s), 1916 (s)

Mass Spec (m/z): 223 (M⁺-Cl-2CO)

Elemental: MnC₁₁H₁₆N₂O₃Cl found (calc) C: 41.65 (41.99), H: 5.25 (5.13), N: 8.70 (8.90), Cl: 11.59 (11.27)

CORM-332 [MnCl(CO)₃(1,10-phenanthroline-5,6-dione)]

115 mg (0.5 mmol) of [MnCl(CO)₅] and 105 mg (0.5 mmol) of 1,10-phenanthroline-5,6-dione, dpq, were refluxed together in 10 ml of ether for ˜1½-2 h, under nitrogen. The solution turned dark brown initially, and then a dark precipitate was produced. The system was then cooled to −80° C. to ensure complete precipitation and the product collected by filtration. It was washed several times with cold ether and then dried under vacuum. 160 mg of a dark green/brown solid was obtained. The yield was 83%. M^{r}=384.

¹H NMR (CD₂Cl₂): δ(ppm) 7.82 (br, 1H), 8.63 (br, 1H), 9.46 (br, 1H)

⁵⁵Mn NMR (CD₂Cl₂): δ(ppm) 270 line width 5780 Hz

IR (THF) ν(cm⁻¹): 2028 (vs), 1941 (s), 1918 (s)

Mass Spec (m/z): 349 (M⁺-Cl)

Elemental: MnC₁₅H₈N₂O₃Cl found (calc) C: 45.35 (46.84), H: 1.67 (1.57), N: 7.15 (7.28), Cl: 9.25 (9.22).

CORM-333 [Mn(CO)₄(2,2′-bipyridine)][BF₄] [10]

113 mg (0.3 mmol) of [MnBr(CO)₃(2,2′-bipyridine)] (CORM-328) and 58 mg (0.3 mmol) of AgBF₄ were stirred together in 10 ml of dry THF under a CO atmosphere for ˜3-4 h. Completion of the reaction was confirmed by IR. The AgBr precipitate was then removed by filtration and 10 ml of pentane was added. Cooling the system to −78° C. resulted in an ‘oily’ precipitate. Hence all solvent was removed and the residue dissolved in the minimum mount of CH₂Cl₂, hexane was added and the system was placed in the freezer overnight. This produced a precipitate that was collected by filtration, washed with hexane and then dried under vacuum. 68 mg of a yellow solid was produced. The yield was 55%. M^{r}=410. (m/z) (—BF₄) 323.

⁵⁵Mn NMR (CD₂Cl₂): δ (ppm) −289 line width 1940 Hz

IR (CH₂Cl₂) ν(cm⁻¹): 2127 (w), 2050 (vs), 1938 (w), 1947 (vs)

Mass Spec (m/z): 323 (M⁺), 295 (M⁺-CO), 239 (M⁺-3CO), 211 (M⁺-4CO).

### CORM-334 [Choline][Mn₂(CO)₆Cl₃] [3]

450 mg (1.95 mmol) of Mn(CO)₅Cl and 223 mg (1.60 mmol) of choline chloride were refluxed in 20-25 ml of dry DCM (dichloromethane), under argon for 1.5 hrs. After being allowed to cool, a further 20 ml of DCM was added to ensure that all of the product had dissolved. It was then filtered, and hexane added. However, this resulted in an “oiling” out of the product. Hence all the solvent was removed on rotary evaporator.

After several attempts, some solid precipitate was formed by recrystallisation from DCM/hexane at −18° C.

120 mg of a yellow/orange solid was obtained. Yield was 25%. Mr=488.47

¹⁷O NMR (CD₂Cl₂): δ(ppm) 384.9 (CO)

IR (CH₂Cl₂) ν(cm⁻¹): 2026 (s), 1938 (s), 1929 (s, sh)

Elemental: C₁₁H₁₄Cl₃Mn₂NO₇ found (calc) C: 27.35 (27.05), H: 4.17 (2.89), N: 4.18 (2.87), Cl: 23.40 (21.77)

Based on the preliminary analytical data, the product was initially identified as [Choline][Mn(CO)₄Cl₂]. However, additional analysis, particularly X ray crystal structure analysis, has revealed that the product has the title structure. The structure [Mn₂(CO)₆Cl₃]⁻ has also been reported. See A. Sieker, A. J. Blake and B. F. G. Johnson, “New mixed carbonyl-nitro and -nitrito complexes of manganese and rhenium,” J. Chem. Soc., Dalton Trans., 1996, 1419-27.

### CORM-338 [Me₃NCH₂CH₂OH][Mn(CO)₄I₂]

450 mg (1.40 mmol) of [Mn(CO)₅I] and 301 mg (1.30 mmol) of choline iodide were stirred in 15 ml of methanol at 55° C. for 36 h. (The IR Spectrum recorded after 2 h showed a significant amount of starting material remained).

Following this, the solvent was removed on a rotary evaporator to give a yellow/brown ‘oily’ solid. This residue was dissolved in DCM, filtered, and then diethyl ether added. This precipitated out a white solid (presumably unreacted choline iodide) which was filtered off. Solvent was then removed on a rotary evaporator and the residue again dissolved in DCM. A little hexane was then added. However, this resulted in the product separating as an oil. Hence all the solvent was removed on a rotary evaporator and the resulting semi-solid residue washed twice with diethyl ether. This produced a solid product that was dried under vacuum.

405 mg (0.771 mmol) of an orange/brown solid was obtained. M^{r}=524.96.

Yield was 59%.

¹H NMR (CD₂Cl₂): δ(ppm) 3.11 (br, OH 1H), 3.35 (br, CH₃ 9H), 3.68 (br, CH₂ 2H), 4.22 (br, CH₂ 2H)

¹³O NMR (CD₂Cl₂): δ(ppm) 55.29 (t {J=3.9 Hz}, CH₃), 56.35 (CH₂), 68.16 (t {J=2.8 Hz}, CH₂), 213.26 (CO), 221.91 (CO)

¹⁷O NMR (CD₂Cl₂): δ(ppm) 377.3 (CO), 379.4 (CO)

⁵⁵Mn NMR (CD₂Cl₂): δ(ppm) −863 line width 6650 Hz

IR (CH₂Cl₂) ν(cm⁻¹): 2077 (s), 2002 (vs), 1984 (s), 1942 (s)

Mass Spec (m/z): 421 (MO), 393 (M⁻-CO), 365 (M⁻-2CO), 337 (M⁻-3CO), 309 (M⁻-4CO)

Elemental: MnC₉H₁₄NO₅I₂ found (calc) C: 20.62 (20.59), H: 2.55 (2.69), N: 2.57 (2.67), I: 48.61 (48.35)

### [Mn(CO)₅(SO₃CF₃)] [11] Used to Make CORMs 349, 369, 370, 371, 376, 377, 378, 379

420 mg (1.53 mmol) of [MnBr(CO)₅] and 490 mg (1.90 mmol) of Ag(SO₃CF₃) were stirred together in 20 ml of dry CH₂Cl₂ under nitrogen for ˜3 h, in the dark (flask wrapped in foil). The reaction was monitored by IR. After this time it was evident that all the [MnBr(CO)₅] had reacted, and so AgBr and excess Ag(SO₃CF₃) were removed by filtration (sinter+filter aid).

The yellow filtrate was then placed on a rotary evaporator in a foil wrapped flask in order to reduce the volume. Hexane was then added and the product taken to dryness on the rotary evaporator. 462 mg of a yellow solid was obtained. The yield was 88%. Note, the product is light sensitive. M^{r}=344.

¹³C. NMR (CD₂Cl₂): δ(ppm) 118.9 (q {J=318 Hz}, CF₃), 202.4 (broad, CO)

¹⁷O NMR (CD₂Cl₂): δ(ppm) 389.4 (eq. CO's), 405.2 (ax. CO)

⁵⁵Mn NMR (CD₂Cl₂): δ(ppm) −228 line width 4200 Hz

IR (CH₂Cl₂) ν(cm⁻¹): 2158 (w), 2073 (vs), 2020 (s)

Mass Spec (m/z): 208 (M⁺-CO), 152 (M⁺-3CO).

### [NMe₄][Acetate] [12] Used in CORM-349

This is commercially available, but earliest reference found in [12].

247 mg (4.11 mmol) of acetic acid and 1.50 g (4.11 mmol) of [NMe₄][OH] (25 wt. % soln. in MeOH) were stirred in 10 ml of methanol for 4 hrs at 40° C. Following this, the solution was filtered and then the solvent removed on rotary evaporator to give a viscous oil, in which solid was starting to form. The last traces of solvent were removed under high vacuum to leave a white solid, which was washed with ether and then dried under vacuum.

480 mg of a white solid were produced. Yield was 87.6%.

### CORM-349 [Me₄N][(OC)₃Mn(μ-OCOCH₃)₃Mn(CO)₃]

150 mg (0.436 mmol) of Mn(CO)₅(SO₃CF₃) and 116 mg (0.872 mmol) of [Me₄N][acetate] were stirred in 8 ml of dry THF and 2 ml of methanol, under argon at 50-55° C. for 3 hrs. During this time the colour of the solution went a little darker yellow/orange.

Following this, the solvent was removed on rotary evaporator to give a yellow/orange semi-solid residue. This was crystallised from DCM/Ether at −18° C. to give a yellow crystalline product (123 mg, 0.232 mmol). Yield was 100%.

Based on the preliminary IR data, the product was initially identified as [Me₄N][(Mn(CO)₄(OAc)₂]. However, additional analysis, particularly X ray crystal structure analysis, has revealed that the product has the title structure. Furthermore, mass spectral data also supports a product having more than one Mn atom.

Mr=529.21.

¹H NMR (CD₂Cl₂): δ(ppm) 2.29 (s, acetate CH₃ 6H), 3.33 (s, NMe₄ 12H)

¹³C NMR (CD₂Cl₂): δ(ppm) 23.55 (acetate CH₃), 56.34 (NMe₄), 176.15 (C═O), 224.20 (CO)

¹⁷O NMR (CD₂Cl₂): δ(ppm) 388.6 (CO)

IR (CH₂Cl₂) ν(cm⁻¹): 2027 (s), 1930 (vs)

Mass Spec (ES⁻) (m/z): 455 ([Mn₂(CO)₆(OAc)₃]⁻); 315 ([Mn₂(CO)(OAc)₃]⁻ or [Mn2(CO)₄(OAc)(OH)₂]⁻); 257 ([Mn(CO)₃(OAc)₂]⁻)

Elemental: C₁₆H₂₁Mn₂NO₁₂ found (calc) C: 36.80 (36.31), H: 4.90 (4.00), N: 3.60 (2.65)

The dimeric structure of the anion has been established by x-ray crystallography.

### [NMe₄]₂[Malonate] Used in CORM-350

428 mg (4.11 mmol) of malonic acid and 1.50 g (4.11 mmol) of [NMe₄][OH] (25 wt. % soln. in MeOH) were stirred in 9 ml of methanol, under argon at 40° C. for 4 hrs. Following this, the solution was filtered and then the solvent removed on rotary evaporator to give a ‘damp’ white solid. This was washed with ether and then dried under vacuum.

712 mg of a white solid were produced. Yield was 97.8%.

### CORM-350 [Me₄N][Mn(CO)₄(Malonate)]

150′ mg (0.546 mmol) of Mn(CO)₅Br and 117 mg (6.00 mmol) of AgBF₄ were stirred together in 8 ml of dry THF under argon for ˜2 hrs. During this time the colour of the solution became more yellow and a dark coloured precipitate was formed. The solution of ‘[Mn(CO)₅(THF)][BF₄]’ was filtered through celite into a stirred. THF suspension of 137 mg (0.546 mmol) of [Me₄N][malonate].

The system was stirred in the dark overnight, after which only a dark coloured precipitate was present, and a yellow solution. IR showed that no pentacarbonyl starting material remained (i.e IR recorded after 2 hrs showed the presence of pentacarbonyl). After filtering, the solvent was removed on rotary evaporator, and the product washed several times with ether.

78 mg of a dark yellow solid was obtained. Yield 38.8%.

### CORM-363 [Mn(CO)₄Br(O₂CCH₂CO₂H)][NMe₄]

150 mg (0.546 mmol) of Mn(CO)₅Br and 95 mg (0.535 mmol) of [Me₄N][O₂CCH₂CH₂CO₂H)] were stirred together in 12 ml MeOH, under argon at 50° C. overnight. Following this it was filtered and then the solvent removed on rotary evaporator to give a ‘damp’ yellow solid. This was washed with ether and then dried under vacuum.

196 mg of a yellow solid were produced. Yield was 86.4%.

### [NMe₄][O₂CCH₂CH₂CO₂H] [13] Used in CORM-364

486 mg (4.11 mmol) of malonic acid and 1.50 g (4.11 mmol) of [NMe₄][OH] (25 wt. % soln. in MeOH) were stirred in 9 ml of methanol, under argon at 35° C. overnight. Following this, the solution was filtered and then the solvent removed on rotary evaporator to give a white solid. This was washed with a little acetone and then ether and then dried under vacuum.

743 mg of a white solid were produced. Yield was 94.4%.

### CORM-364 [Mn(CO)₄Br(O₂CCH₂CH₂CO₂H)][NMe₄]

100 mg (0.364 mmol) of Mn(CO)₅Br and 70 mg (0.364 mmol) of [Me₄N][O₂CCH₂CH₂CO₂H)] were stirred together in MeOH/DCM (7:3), under argon at 40-45° C. overnight. Following this it was filtered and then the solvent removed on rotary evaporator to give a ‘damp’ yellow solid. This was washed with DCM and ether and then dried under vacuum.

130 mg of a yellow solid were produced. Yield was 81.6%.

### [(15-Crown-5)Na][Br] Used in CORM-365

1.070 g (4.86 mmol) of 15-Crown-5 (commercially available) and 500 mg (3.78 mmol) of NaBr were stirred together in 15 ml: of methanol at 50° C. for 3 hrs. Following this, the solvent was removed on rotary, evaporator to give a solid product that was washed several times with ether and then dried under vacuum.

1.317 g of a white solid was obtained. Yield was 83.9%.

### CORM-365 [Mn(CO)₄Br₂][(15-Crown-5)Na] [14], [3]

200 mg (0.727 mmol) of Mn(CO)₅Br and 219 mg (0.678 mmol) of [(15-Crown-5)Na]Br were stirred together in 10 ml of MeOH, under argon for 24 hrs at 50-55° C. Following this the solvent was removed on rotary evaporator to give an orange/brown oily residue. This was dissolved in 20 ml of DCM, filtered, and then hexane added. However, this resulted in an oiling out of the product.

Hence, all the solvent was removed on rotary evaporator to give an oily residue again. Ether was added and then removed on rotary evaporator to give a residue that had started to solidify. A small amount of ether was then added, and the solid product isolated. This was washed with pentane and then dried under vacuum.

256 mg of an orange solid was obtained. Yield was 66.2%.

### CORM-368 Mn(CO)₄(η²-S₂CNEt₂) [15], [16]

150 mg (0.436 mmol) of Mn(CO)₅(SO₃CF₃) and 98 mg (0.436 mmol) of Na[S₂CNEt₂].3H₂O (commercially available) were stirred in 8-9 ml of acetone, under argon at 35° C. for 4 hrs. Solvent was then removed on rotary evaporator to leave a yellow residue, which was extracted with ether. Removal of solvent gave a yellow solid. This was recrystallised from (a) pentane at −18° C. for 2 days which gave 33 mg of a yellow solid, and (b) hexane at −78° C. for ˜1 hr which gave 55 mg of a pale yellow solid. A further 29 mg were obtained from the residue.

Combined yield was 117 mg, which was 85.1%.

¹H NMR (CD₂Cl₂): δ(ppm) 1.28 (t {J=7.2 Hz}, CH₃ 3H), 3.75 (q {J=7.0 Hz}, CH₂ 2H)

¹³C NMR (CD₂Cl₂): δ(ppm) 11.99 (CH₃), 43.99 (CH₂), 206.37 (CS₂), 211.67 (CO) 216.92 (CO)

¹⁷O NMR (CD₂Cl₂): δ(ppm) 367.3 (CO), 380.0 (CO)

⁵⁵Mn NMR (CD₂Cl₂): δ(ppm) −1031 line width 2690 Hz

IR (CH₂Cl₂) ν(cm⁻¹): 2086 (m), 2007 (vs), 1990 (s), 1947 (s)

Mass Spec (m/z): 203 (M⁺-400)

Elemental: MnC₉H₁₀NS₂O₄ found (calc) C: 34.35 (34.29), H: 3.15 (3.20), N 4.41: (4.44), S: 20.56 (20.34).

### CORM-369 (Choline][Mn(CO)₄I₂] [3]

450 mg (1.40 mmol) of Mn(CO)₅I and 301 mg (01.30 mmol) of choline iodide were stirred in 15 ml of methanol at 55° C. for 36 hrs. (IR recorded after 2 hrs showed a significant amount of starting material remaining).

Following this, the solvent was removed on rotary evaporator to give a yellow/brown ‘oily’ solid. This residue was dissolved in DCM, filtered, and then ether added. This crashed out a white solid (presumably unreacted choline iodide) which was filtered off. Solvent was then removed on rotary evaporator and the residue again dissolved in DCM. A little hexane was then added. However, this resulted in the product crashing out as an oil. Hence all the solvent was removed on rotary evaporator and the resulting semi-solid residue washed twice with ether. This produced a solid product that was dried under vacuum.

405 mg of an orange/brown solid was obtained. Yield was 59.3%.

¹H NMR (CD₂Cl₂): δ(ppm) 3.35 (br, NMe₃), 3.69 (br, CH₂), 4.18 (br, CH₂)

¹³C NMR (CD₂Cl₂): δ(ppm) 54.0 (NMe₃), 56.6 (CH₂), 68.5 (CH₂), 211.6 (CO), 219.8 (CO),

IR (CH₂Cl₂) ν(cm⁻¹): 2092 (w), 2015 (vs), 1989 (s), 1943 (s)

Mass Spec (m/z): 215 (M⁻-4CO) (1:2:1 ratio of peaks observed, i.e. ⁷⁹Br/⁸¹Br).

### [Me₄N][Boc-alanate]. Used in CORM-370

778 mg (4.11 mmol) of Boc-alanine and 1.50 g (4.11 mmol) of [NMe₄][OH] (25 wt. % soln. in MeOH) were stirred in 10 ml of methanol, under argon at 35° C. overnight. Following this, the solution was filtered and then the solvent removed on rotary evaporator to give a ‘damp’ white solid. This was washed with a little acetone and then ether and then dried under vacuum.

1.007 g of a white solid were produced. Yield was 93.4%.

### CORM-370 [Me₄N][Mn₂(CO)₆(Boc-alanate)₃]

150 mg (0.436 mmol) of Mn(CO)₅(SO₃CF₃) and 220 mg (0.837 mmol) of [Me₄N][Boc-alanate] were stirred in 8 ml of dry THF and 2 ml of methanol, under argon at 50-55° C. for 3 hrs. During this time the colour of the solution went a little darker yellow/orange.

Following this, the solvent was removed on rotary evaporator to give a yellow/orange semi-solid residue. This was dissolved in DCM, filtered through celite to remove [Me₄N][SO₃CF₃] by-product, and then ether added to precipitate the product. However, a gel-like precipitate was formed, so it was collected on celite, washed several times with ether, and then washed through the celite with DCM. Removal of solvent on rotary evaporator and then drying under vacuum gave 209 mg of an orange/yellow solid product. Yield was 100%.

¹H NMR (CD₂Cl₂): δ(ppm) 0.92 (s, alanine CH₃), 1.55 (s, ^{t}Bu CH₃), 3.47 (s, v. broad, NMe₄), 4.09 (alanine CH), 8.52 (s, v. broad, NH). All signals very broad. Spectrum not very useful.

¹³C NMR (CD₂Cl₂): δ(ppm) 19.5 (alanine CH₃), 27.9 (^{t}Bu CH₃), 56.7 (NMe₄), 78.6 (alanine CH), 83.4 (^{t}Bu CMe₃), 155.5 (C═O), 160.3 (C═O), 222.1 (CO). All signals are broad.

¹⁷O NMR (CD₂Cl₂): δ(ppm) 385.5 (CO), 386.8 (CO)

IR (CH₂Cl₂) ν(cm⁻¹): 2032 (s), 1919(vs), 1747 (w), 1701 (m), 1630 (s)

Mass Spec (m/z): 653 ([Mn₂(CO)₆(Boc-alanate)₂-H⁻]⁻); 515 ([Mn(CO)₃(Boc-alanate)₂]⁻; 515 ([Mn(CO)₃(Boc-alanate)-H⁺]⁻)

Elemental: C₃₄H₅₄Mn₂N₄O₁₈ found (calc) C: 46.31 (44.55), H: 7.00 (5.95), 6.83 (6.11).

Based on the preliminary analytical data, the product was initially identified as [Me₄N][Mn(CO)₄(Boc-alanate)₂]. However, additional analysis has revealed that the product has the title structure.

[NMe₄][thioacetate] Used in CORM-371

Commercially available.

418 mg (5.49 mmol) of thioacetic acid and 2.00 g (5.49 mmol) of [NMe₄][OH] (25 wt. % soln. in MeOH) were stirred in 12 ml of methanol overnight at 35° C. Following this, the system was filtered and then the solvent removed on rotary evaporator to give an off-white solid residue. This was washed several times with ether and then dried under high vacuum.

782 Mg of an off-white solid was obtained. Yield was 95.4%.

CORM-371 [Me₄N][Mn(CO)₄(thioacetate)₂]

150 mg (0.436 mmol) of Mn(CO)₅(SO₃CF₃) and 128 mg (0.857 mmol) of [Me₄N][thioacetate] were stirred in 8 ml of dry THF and 2 ml of methanol, under argon at 50-55° C. for 4.5-5 hrs. During this time the colour of the solution went a little darker yellow/orange.

Following this, the solvent was removed on rotary evaporator to give a yellow/orange semi-solid residue. This was crystallised from DCM/Ether at −18° C. to give 91 mg of a yellow crystalline product. Yield was 55.7%. M^{r}=391.34

¹H NMR (CD₂Cl₂): δ(ppm) 2.41 (s, thioacetate CH₃ 6H), 3.35 (s, NMe₄ 12H)

¹³C NMR (CD₂Cl₂): δ(ppm) 34.78 (thioacetate CH₃), 56.27 (t {J=3.7 Hz}, NMe₄), 205.79 (C═O)

¹⁷O NMR (CD₂Cl₂): ε(ppm) 369.2 (CO), 371.2 (CO)

⁵⁵Mn NMR (CD₂Cl₂): δ(ppm) −1318 line width 1440 Hz

IR (CH₂Cl₂) ν(cm⁻¹): 2073 (m), 1992 (vs), 1976 (s, sh), 1934 (s)

Mass Spec (m/z): 317 (M⁻), 289 (M⁻-CO)

Elemental: MnC₁₂H₁₈NS₂O₆ found (calc) C: 37.72 (36.83), H: 4.63 (4.64), N: 3.83 (3.58), S: 16.06 (16.39) [Sample contains some DCM]

### CORM-376 [K][(OC)₃Mn(μ-OCOCH₃)₃Mn(CO)₃]

250 mg (0.727 mmol) of Mn(CO)₅(SO₃CF₃) and 143 mg (1.45 mmol) of potassium acetate were stirred together in 10 ml of MeOH, under argon for 15 hrs at 50° C. Following this the solvent was removed or rotary evaporator to give a yellow residue. This was dissolved: in ethyl acetate and then filtered through: celite to remove K[SO₃CF₃] by-product. Ether was then added to precipitate the product. It was collected on a sinter, washed several times with ether and then dried under vacuum.

160 mg, of a yellow solid obtained. Yield 56.7%.

Based on the preliminary analysis data, the product was initially identified as [K][(Mn(CO)₄(OAc)₂]. However, additional analysis, particularly X ray crystal structure analysis on the related: CORM-349 compound, has revealed that the product has the title structure.

¹H NMR (CD₃CN): δ(ppm) 2.37 (s, CH₃)

¹³C NMR (CD₃CN): δ(ppm) 22.4 (CH₃), 175.3 (C═O), 223.7 (CO)

¹⁷O NMR (CD₃CN): δ(ppm) 387.9 (CO)

IR (MeCN) ν(cm⁻¹): 2028 (s), 1931 (vs), 1919 (s, sh), 1661 (m, C═O)

Mass Spec (ES⁻) (m/z): 455 ([Mn₂(CO)₆(OAc)₃]⁻); 315 ([Mn₂(CO)(OAc)₃]⁻ or [Mn₂(CO)₄(OAc)(OH)₂]⁻); 257 ([Mn(CO)₃(OAc)₂]⁻)

Elemental: Cl₂H₂KMn₂O₁₂ found (calc) C: 29.90 (29.17), H: 2.69 (1.84)

### Na[S₂CN{CH₂CH₂OH}₂] [18] Used in CORM-378

2.218 g (55.5 mmol) of powdered NaOH was dissolved in 40 ml of EtOH, under argon. [Note, this took a while, and required some heating). A solution of 5.830 g (55.5 mmol) of diethanolamine was then added to this.

With cooling in an ice/water bath, and continuous stirring, a solution of 4.433 g (58.2 mmol) of CS₂ in 12 ml of ether was added drop-wise. This resulted in the immediate formation of a pale yellow/green colour in the solution. After complete addition, the system was stirred at room temperature for an hour.

Following this, ether was added but this resulted in the product crashing out as an oily solid. Hence the supernatant was removed, and the product dissolved in warm ethanol. This was then poured into the supernatant (which was predominantly ether) and then as it cooled the product precipitated out as a white crystalline solid. It was then cooled in an ice/water bath to complete precipitation. The product was collected on a sinter, washed several times with ether and then dried under vacuum.

7.715 g of a very slightly off-white solid was obtained. Yield was 68.5%.

### CORM-378 Mn(CO)₄(η²-S₂CN{CH₂CH₂OH}₂)

### Method (a)

120 mg (0.349 mmol) of Mn(CO)₅(SO₃CF₃) and 71 mg (0.349 mmol) of Na[S₂CN{CH₂CH₂OH}₂] were stirred in 7 ml of acetone, under argon at 45° C. for 2 hrs. Solvent was then removed on rotary evaporator to leave a yellow residue, which was extracted with ether. Removal of solvent gave a yellow oily product. This was recrystallised from ether/pentane at −18° C. to give 65 mg of a yellow crystalline solid. Reduction of solvent volume and addition of more pentane gave a second crop of 36 mg. These were dried under vacuum.

Combined yield was 101 mg, which was 83.4%.

### Method (b)

300 mg (1.09 mmol) of Mn(CO)₅Br and 222 mg (1.09 mmol) of Na[S₂CN{CH₂CH₂OH}₂) were stirred in 15 ml of acetone, under argon at 60° C. for 2 hrs. Solvent was then removed on rotary evaporator to leave a yellow residue, which was extracted with ether. Removal of solvent gave a yellow oily product. This was recrystallised from ether/pentane at −18° C. to give 223 mg of a yellow crystalline solid. Reduction of solvent volume and addition of more pentane gave a second crop of 71 mg. These were dried under vacuum.

Combined yield was 294 mg, which was 77.7%.

¹H NMR (CD₂Cl₂): δ(ppm) 4.00 (s, both CH₂)

¹³C NMR (CD₂Cl₂): δ(ppm) 54.27 (CH₂), 60.25 (CH₂), 210.10 (CS₂), 211.29 (CO), 216.38 (CO)

¹⁷O NMR (CD₂Cl₂): δ(ppm) 368.4 (CO), 380.9 (CO)

⁵⁵Mn NMR (CD₂Cl₂): δ(ppm) −1001 line width 5060 Hz

IR (CH₂Cl₂) ν(cm⁻¹): 2087 (m), 2008 (vs), 1994 (s), 1950 (s)

Mass Spec (m/z): 347 (M⁺), 291 (M⁺-2CO), 263 (M⁺-3CO), 235 (M⁺-4 CO)

Elemental: MnC₉H₁₀NS₂O₆ found (calc) C: 31.15 (31.13), H: 2.76 (2.90), N: 3.92 (4.03), S: 18.29 (18.47).

[NMe₄[benzoate]⁸ Used for CORM-379.

568 mg (4.11 mmol) of benzoic acid and 1.50 g (4.11 mmol) of [NMe₄][OH] (25% soln. in MeOH) were stirred in 8 ml of methanol at 45° C. for 4 h. Following, this, the solution was filtered and then the solvent removed on a rotary evaporator to give a ‘damp’ white solid. This was placed under high vacuum for several hours, to complete solidification. It was then washed with a little acetone and diethyl ether. The resulting product was then dried under vacuum.

601 mg (3.08 mmol) of a white solid was produced. M^{r}=195.26. Yield 75%. Commercially available. See also A. Pacheco, B. R. James, S. J. Rettig, Inorg. Chem., 1995, 34, 3477.

CORM-379 [Me₄N][Mn₂(CO)₆(benzoate)₃]

150 mg (0.436 mmol) of [Mn(CO)₅(SO₃CF₃)] and 169 mg (0.863 mmol) of [Me₄N][benzoate] were stirred in 8 ml of dry THF and 2 ml of methanol, under argon at 50-55° C. for 3 h. During this time the colour of the solution went a little darker yellow/orange.

Following this, the solvent was removed on a rotary evaporator to give a yellow/orange semi-solid residue. This was crystallised from DCM/diethyl ether/pentane at −18° C. to give a yellow solid.

119 mg (0.246 mmol) of product obtained. M^{r}=483.35. Yield 57%.

¹H NMR (CD₂Cl₂): δ(ppm) 3.3 (br, NMe₄), 7.31 (br, Ph), 7.43 (br, Ph), 7.92 (br, Ph)

¹³C NMR (CD₂Cl₂): δ(ppm) 56.98 (NMe₄), 127.84 (meta Ph), 128.73 (ortho Ph), 130.86 (para Ph), 135.44 (ipso Ph), 177.7 (C═O), 224.08 (CO)

¹⁷O NMR (CD₂Cl₂): δ(ppm) 387.9 (CO)

IR (CH₂Cl₂) ν(cm⁻¹): 2026 (s), 1913 (vs), 1606 (m)

Mass Spec (m/z): 641 ([Mn₂(CO)₆(O₂CPh)₃]⁻); 537 ([Mn₂(CO)₆(O₂CPh)₂(OH)]⁻); 433 ([Mn₂(CO)₆(O₂CPh)(OH)₂]⁻); 381 ([Mn(CO)₃(O₂CPh)₂]⁻)

Elemental: Mn₂C₃₁H₂₇NO₁₂ found (calc) C: 50.54 (52.04), H: 5.06 (3.80), N: 3.12 (1.96).

### CORM-388 [Mn(CO)₄(S₂COEt)].

150 mg (0.546 mmol) of [Mn(CO)₅Br] and 88 mg (0.546 mmol) of K[S₂COEt] were stirred in 8-9 ml of acetone, under argon at 55° C. for ˜1.5 h. Solvent was then removed on a rotary evaporator to leave a yellow residue, which was extracted with diethyl ether. Removal of solvent gave a yellow solid. Attempted recrystallisation from hexane at −18° C. for 2 d did not result in any solid. Hence it was cooled to −78° C. for ˜1 h which resulted in precipitation of the product.

120 mg (0.416 mmol) of an orange solid was produced. M^{r}=315.25. Yield 76%.

¹H NMR (CD₂Cl₂): δ(ppm) 1.48 (t {J=7.1 Hz}, CH₃ 3H), 4.63 (q {J=7.0 Hz}, CH₂ 2H)

¹³C NMR (CD₂Cl₂): δ(ppm) 13.5 (CH₃), 68.6 (CH₂), 226.7 (CS₂), 210.2 (CO), 216.1 (broad, CO)

¹⁷O NMR (CD₂Cl₂): δ(ppm) 371.9 (CO), 383.4 (CO)

⁵⁵Mn NMR (CD₂Cl₂): δ(ppm) −964 line width 3040 Hz

IR (CH₂Cl₂) ν(cm⁻¹): 2094 (m), 2015 (vs), 2003 (s), 1959 (s)

Mass Spec (m/z): 288 (M⁺)

Elemental: MnC₇H₅S₂O₅ found (calc) C: 29.36 (29.17), H: 1.26 (1.75), S: 21.94 (22.25)

For further details see H. Laufen, B. Meyn, K. G. Steinhaeuser, D. Vogel and R. Kramolowsky, J. Organomet. Chem., 1976, 112, C34.

### Na[S₂CNMe(CH₂CO₂Na)] Used in CORM-401

Commercially available or may be prepared according to the method described in J. A. Beatty, M. M. Jones, D. J. Wilson and L Ma, Chem. Res. Toxicol., 1992, 5, 568.

### CORM-401 [Mn(CO)₄(S₂CNMeCH₂CO₂H)]

500 mg (0.0018 mol) of Mn(CO)₅Br and 420 mg (0.0018 mol) of NaS₂CN(CH₃)(CH₂COONa) were stirred in 36-40 ml of methanol, under argon at 40° C. for 4 hrs. The solvent was then removed to leave a yellow solid. An aqueous solution of the yellow solid was acidified to pH 2 with 0.1M H₂SO₄ to produce yellow precipitate that was washed with H₂SO₄ and dried:

Combined yield was 407 mg (0.0012 mol), which was 66.7%. M^{r}=331.21

¹H NMR (CD₂Cl₂): δ(ppm) 3.39 (CH₃ 3H), 4.62 (CH₂ 2H)

¹³C NMR (CD₂Cl₂): δ(ppm) 38.16 (CH₂), 51.39 (CH₃), 171.44 (CO₂H), 211.36 (CS₂), 210.97 216.51 (br, CO).

IR (CH₂Cl₂)(cm⁻¹): 2088 (m); 2010 (vs), 1994 (s), 1951: (s)

Mass Spec (m/z): 331 (M⁺), 275 (M⁺-2 CO's), 247 (M⁺-3 CO's), 219 (M⁺-4 CO's).

Elemental MnC₈H₆NO₆S₂found (calc) C: 29.01 (29.01), H: 1.99 (1.83), N: 3.91 (4.23), S: 19.16 (19.36).

### CORM-402. [Mn(CO)₄{S₂P(OEt)₂}]

In a Schlenk tube, Mn(CO)₅Br (550 mg, 2 mmol) was placed with a stirrer bar under argon. Diethyl ether (30 ml) was added to give a yellow solution. Then KS₂P{OEt}₂ (450 mg, 2 mmol) in diethyl ether (10 ml) was added dropwise and the solution allowed to stir overnight.

The next day the solution was filtered and the solvent was removed trap-to-trap to afford a yellow residue. This was chromatographed on a Florosil column (20×1 cm) using petrol as eluent. Removal of the solvent afforded the pure product as a bright yellow solid.

Yield: 468 mg (66%). Mr=352.21

¹H NMR (CD₂Cl₂): (ppm) 4.05 (m, 2H), 1.27 (t, 3H)

³¹P NMR (CD₂Cl₂): (ppm) 91.0

¹³C NMR (CD₂Cl₂): (ppm) 15.6 (J=8 Hz), 64.0 (J=6 Hz) 208.9, 216.8 (br, CO)

IR (CHCl₃)(cm⁻¹): 2095 (m), 2020 (vs), 2003 (s), 1965 (s).

See also R. L. Lambert and T. A. Manuel, Inorg. Chem., 1966, 5, 1287.

### REFERENCES FOR PRIOR ART SECTION

1 Piantadosi C A. Toxicity of carbon monoxide: hemoglobins vs. histotoxic mechanisms. In: Carbon monoxide. (Edited by Penney D G). 1996; Chapter 8.
2 Sjostrand T. Endogenous formation of carbon monoxide in man under normal and pathological conditions. Scan J Clin Lab Invest 1949; 1: 201-14.
3 Coburn R F, Blakemore W S, Forster R E. Endogenous carbon monoxide production in man. J Clin Invest 1963; 42: 1172-8.
4 Coburn R F, Williams W J, Forster R E. Effect of erythrocyte destruction on carbon monxide production in man. J Clin Invest 1964; 43: 1098-103.
5 Coburn R F, Williams W J, Kahn S B. Endogenous carbon monoxide production in patients with hemolytic anemia. J Clin Invest 1966; 45: 460-8.
6 Sjostrand T. The formation of carbon monoxide by in vitro decomposition of haemoglobin in bile pigments. Acta Physiol Scand 1952; 26: 328-33.
7 Coburn R F, Williams W J, White P, Kahn S B. The production of carbon monoxide from hemoglobin in vivo. J Clin Invest 1967; 46: 346-56.
8 Tenhunen R, Marver H S, Schmid R. Microsomal heme oxygenase. Characterization of the enzyme. J Biol. Chem. 1969; 244: 6388-94.
9 Scharf S M, Permutt S, Bromberger-Barnea B. Effects of hypoxic and CO hypoxia on isolated hearts. J Appl Physiol 1975; 39: 752-8.

### REFERENCES FOR EXPERIMENTAL DATA SECTION

1 WO 2005/114161
2 WO 2004/045599
3. Motterlini R, Clark J E, Foresti R, Sarathchandra P, Mann B E and: Green C J. Carbon monoxide-releasing molecules: characterization of biochemical and vascular activities. Circ Res 90: E17-E24, 2002.
4. Sammut I A, Foresti R, Clark J E, Exon D J, Vesely M J J, Sarathchandra P, Green C J and Motterlini R. Carbon monoxide is a major contributor to the regulation of vascular tone in aortas expressing high levels of haeme oxygenase-1. Br J Pharmacol 125: 1437-1444, 1998.

### REFERENCES FOR SYNTHESES SECTION

1 E. W. Abel and G. Wilkinson, J. Chem. Soc. (London), 1959, 1501.
2 M. H. Quick and R. J. Angelici, Inorg. Synth. 1979, 19, 161.
3 R. J. Angelici, Inorg. Chem. 1964, 3, 1099.
4 L. H. Staal, A. Oskam and K. Vrieze, J. Organomet. Chem. 1979, 170, 235.
5 R. H. Reimann, and E. Singleton, J. Chem. Soc. (Dalton) 1973, 841.
6 S. A. Moya, J. Guerrero, R. Pastene, I. Azócar-Guzmán and A. J. Pardey, Polyhedron, 2002, 21, 439.
7 D. Drew, D. J. Darensbourg and M. Y. Darensbourg, Inorg. Chem. 1975, 14, 1579.
8 R. J. Angelici and D. L. Denton, Inorg. Chim. Acta, 1968, 2, 3.
9 (a) D. M. Haddleton, M. C. Crossman, B. H. Dana, D. J. Duncalf, A. M. Heming, D. Kukulj and A. J. Shooter, Macromolecules, 1999, 32, 2110. (b) D. M. Haddleton, D. J. Duncalf, D. Kukulj, M. C. Grossman, S. G. Jackson, S. A. F. Bon, A. J. Clark and A. J. Shooter, Eur. J. lnorg. Chem., 1998, 1799.
10 L. H. Staal, A. Oskam and K. Vrieze, J. Organomet. Chem. 1979, 170, 235.
11 J. Nitschke, S. P. Schmidt and W. C. Trogler, Inorg. Chem. 1985, 24, 1972; S. P. Schmidt, J. Nitschke, W. C. Trogler, S. I. Huckett, and R. J. Angelici, Inorg. Synth., 1989, 26, 113.
12 G. Jander, E. Rusberg, H. Schmidt, Z. Anorg. Allg. Chem., 1948, 255, 238.
13 N. V. Ignat'ev and S. D. Datsenko, Russian J. Electrochem. (Translation of Elektrokhimiya), 1995, 31, 1235-39.
14 El-Kholy and All El-Sayed, Egyptian Journal of Chemistry, 1981, Volume Date 1979, 22, 23; A. Fischer, Zeitschrift fur Kristallographie, 1996, 211, 827;
15 F. A. Cotton and J. A. McCleverty, Inorg. Chem. 1964, 3, 1398.
16 D. Rehder, R. Kramolowsky, K. G. Steinhauser, U. Kunze and A. Antoniadis, Inorg. Chim. Acta, 1983, 73, 243.
17 D. De Filippo, P. Deplano, F. Devillanova, E. F. Trogu and G. Verani, J. Organomet. Chem., 1973, 38, 560.
18 P. Giboreau and C. Morin, J. Org. Chem., 1994, 59, 1205.
19 J. A. Beatty, M. M. Jones, D. J. Wilson and L. Ma, Chem. Res. Toxicol., 1992, 5, 568.
20 R. L. Lambert and T. A. Manuel, Inorg. Chem., 1966, 5, 1287.

1. A pharmaceutical composition comprising as an active ingredient a compound or ion:
(a) of the formula (I)
Mn(CO)₄XY (I)
wherein X and Y do not occupy trans positions in the molecule relative to each other, and
wherein X and Y are the same or different and
each of X and Y is selected from halogens and monodentate ligands to Mn bonding through one of O and S, or X and Y are together a bidentate ligand to Mn bonding through O, S or both O and S; or
(b) of the formula (III) 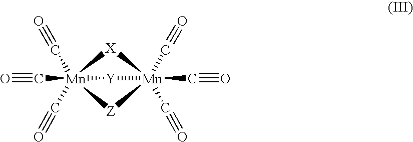
wherein each X, Y and Z is a halogen or a monodentate ligand bonding through O or S, or a bidentate ligand bonding through O, S or both O and S, wherein X, Y and Z are the same or different, and
wherein X, Y and Z do not occupy trans positions relative to each other about either of the two Mn atoms,
or, when (I) or (III) is a compound, a pharmaceutically acceptable salt thereof,
the composition further including, when (I) or (III) is an ion, a pharmaceutically acceptable counter-ion.

2. A pharmaceutical composition according to claim 1, wherein the active ingredient is of formula (I) and
(i) each of X and Y is selected from halogen and 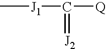
wherein each of J₁ and J₂ is independently selected from O and S and Q is optionally substituted alkyl, alkenyl, aryl, arylalkyl or arylalkenyl, or
(ii) X and Y taken together are a bidentate ligand selected from 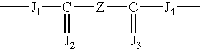
wherein each of J₁, J₂, J₃ and J₄ is independently selected from O and S and Z is optionally substituted alkane-di-yl or alkene-di-yl, or
(iii) X and Y taken together are provided by 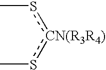
wherein each of R₃ and R₄ is independently selected from H and optionally substituted alkyl, or R₃ and R₄ are together provided by optionally substituted alkane-di-yl or alkene-di-yl having 3 to 6 C atoms or —R₅—O—R₆— wherein each of R₅ and R₆ is optionally substituted alkane-di-yl having 1 to 3 C atoms.

3. A pharmaceutical composition according to claim 2, wherein
Q is alkyl or alkenyl having 1 to 10 C atoms, preferably 1 to 4 C atoms, optionally substituted by one or more of
—COOH, —CSOH; —COOR′; —CONH₂; —CONHR′; —CON(R′)₂; —COR′; —F, —Cl, —Br, I; —CN; —NO₂; —OH; —OR′; —SH; —SR′; —O—CO—R′; —NH₂; —NHR′; —NH(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,
wherein R′ is alkyl or alkenyl of 1 to 6 C atoms,
Z is alkane-di-yl or alkene-di-yl of 1 to 10 C atoms (preferably 1 to 5 C atoms) optionally substituted by any one or more of
—COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —NH(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl, wherein R′ is alkyl or alkenyl of 1 to 6 C atoms, and each of R₃ and R₄ (when not H), R₅ and R₆ is optionally substituted by any one of: —COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —NH(R′)₂; —NH—CO—R′; —NR′—CO—R′; —NR′—SO₂H, —NH—SO₂H; —NR′—SO₂R′, —NR′—SO₂H; —SO₂R; —OSO₂R′; —C₅₋₂₀aryl; —C₁₋₇alkyl-C₅₋₂₀aryl; —C₁₋₇alkenyl-C₅₋₂₀aryl, wherein R′ is alkyl or alkenyl of 1 to 6 C atoms.

4-7. (canceled)

8. The pharmaceutical composition according to claim 1, wherein the active ingredient is of formula (III) and each of X, Y and Z is independently selected from:
(i) 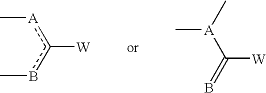
and A and B are independently selected from O and S, and W is optionally substituted alkyl, alkenyl, aryl, arylalkyl, arylalkenyl or W is the group —N(R₃R₄), wherein each of R₃ and R₄, is independently selected from H and optionally substituted alkyl, or R₃ and R₄ are together provided by optionally substituted alkane-di-yl or alkene-di-yl having 3 to 6 C atoms or —R₅—O—R₆—
wherein each of R₅ and R₆ is optionally substituted alkane-di-yl having 1 to 3 C atoms; and
(ii) 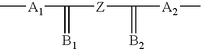
wherein each of A₁, A₂, B₁, and B₂ is independently selected from O and S, and Z is optionally substituted alkane-di-yl or alkene-di-yl.

9. (canceled)

10. The pharmaceutical composition according to claim 1, wherein the active ingredient is of formula (III) and each of X, Y or Z is

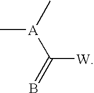

11-14. (canceled)

15. The pharmaceutical composition according to claim 1, comprising an ion having the formula selected from the group: [(OC)₃Mn(μ-OCOCH₃)₃Mn(CO)₃]⁻, [Mn₂(CO)₆(Boc-Alanine)₃]⁻ and [Mn₂(CO)₆Cl₃]⁻.

16. (canceled)

17. Use of a compound or ion of formula (I) or formula (III) as defined in claim 1, in medicine.

18. A method of introducing CO into a mammal as a physiologically effective agent, comprising the step of administering a pharmaceutical composition according to claim 1.

19. A method according to claim 18, for stimulating neurotransmission or vasodilation, or for the treatment of any hypertension, radiation damage, endotoxic shock, inflammation, an inflammatory-related disease, hyperoxia-induced injury, apoptosis, cancer, transplant rejection, arteriosclerosis, post-ischemic organ damage, myocardial infarction, angina, haemorrhagic shock, sepsis, penile erectile dysfunction and adult respiratory distress syndrome.

20. A method of treatment of an extracorporeal or isolated organ, comprising contacting the organ with a pharmaceutical composition according to claim 1.

21. A method according to claim 20, wherein the metal carbonyl makes available carbon monoxide (CO) to limit post-ischemic damage.

22-24. (canceled)

25. Use of a compound or ion of the formula (I) or of the formula (III) as defined in claim 1, for stimulating neurotransmission or vasodilation, or for the treatment of any hypertension, radiation damage, endotoxic shock, inflammation, an inflammatory-related disease, hyperoxia-induced injury, apoptosis, cancer, transplant rejection, arteriosclerosis, post-ischemic organ damage, myocardial infarction, angina, haemorrhagic shock, sepsis, penile erectile dysfunction and adult respiratory distress syndrome.

26. Use of a compound according to claim 25, for treatment of an isolated organ to limit post-ischemic damage in an isolated organ which is inside or attached to the body but isolated from the blood supply.

27. Use of a compound or ion of formula (I) or of the formula (III) as defined in claim 1, in the manufacture of a medicament for administration by an oral, intravenous, subcutaneous, nasal, inhalatory, intramuscular, intraperitoneal, transdermal or suppository route, for the stimulation of neurotransmission or vasodilation by CO as a physiologically effective agent, or for the treatment of any hypertension, radiation damage, endotoxic shock, inflammation, an inflammatory-related disease, hyperoxia-induced injury, apoptosis, cancer, transplant rejection, arteriosclerosis, post-ischemic organ damage, myocardial infarction, angina, haemorrhagic shock, sepsis, penile erectile dysfunction and adult respiratory distress syndrome.

28. A kit for producing a pharmaceutical solution, comprising a compound or ion of formula (I) or of the formula (III) as defined in claim 1, in solid form and a pharmaceutically acceptable solvent.

29. A compound having an anion of the formula (II):
Mn(CO)₄XY (II)
and a counter-cation,
wherein X and Y do not occupy trans positions in the molecule relative to each other, and
wherein X and Y are the same or different and
(i) each of X and Y is selected from
—O—CO-Q
wherein Q is optionally substituted alkyl, alkenyl, aryl, arylalkyl or arylalkenyl, or
(ii) X and Y taken together are a bidentate ligand selected from 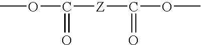
wherein Z is optionally substituted alkane-di-yl or alkene-di-yl.

30. A compound according to claim 29, wherein
Q is alkyl or alkenyl having 1 to 10 C atoms, preferably 1 to 4 C atoms, optionally substituted by one or more of
—COOH, —CSOH; —COOR′; —CONH₂; —CONHR′; —CON(R′)₂; —COR′; —F, —Cl, —Br, —I; —CN; —NO₂ —OH; —OR′; —SH; —SR′; —O—CO—R′; —NH₂; —NHR′; —NH(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl,
wherein R′ is alkyl or alkenyl of 1 to 6 C atoms,
Z is alkane-di-yl or alkene-di-yl of 1 to 10 C atoms (preferably 1 to 5 C atoms) optionally substituted by one or more of
—COOH; —COOR′; —CONH₂; —CONHR′; —CON(R′)₂; —COR′; —F, —Cl, —Br, —I; —CN; —NO₂; —OH; —OR′; —SH; —SR′; —O—CO—R′; —NH₂; —NHR′; —NH(R′)₂; —NH—CO—R′; —NR′—CO—R′; —NR′—SO₂H, —NH—SO₂H; —NR′—SO₂R′, —NR′—SO₂H; —SO₂R′; —OSO₂R′; —C₅₋₂₀aryl; —C₁₋₇alkyl-C₅₋₂₀aryl; —C₁₋₇alkenyl-C₅₋₂₀aryl, wherein R′ is alkyl or alkenyl of 1 to 6 C atoms.

31. (canceled)

32. A compound or ion of the formula (IV): 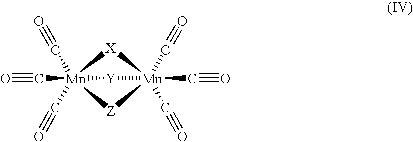
wherein each X, Y and Z is a monodentate ligand bonding through O or S, or a bidentate ligand bonding through O, S or both O and S,
wherein X, Y and Z are the same or different, and
wherein X, Y and Z do not occupy trans positions relative to each other about either of the two Mn atoms.

33. A compound or ion according to claim 32, wherein each of X, Y and Z is independently selected from:
(i) 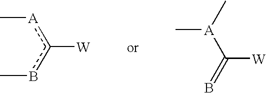
and A and B are independently selected from O and S, and W is optionally substituted alkyl, alkenyl, aryl, arylalkyl, arylalkenyl or W is the group —N(R₃R₄), wherein each of R₃ and R₄ is independently selected from H and optionally substituted alkyl, or R₃ and R₄ are together provided by optionally substituted alkane-di-yl or alkene-di-yl having 3 to 6 C atoms or —R₅—O—R₆— wherein each of R₅ and R₆ is optionally substituted alkane-di-yl having 1 to 3 C atoms; and
(ii) 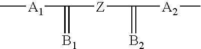
wherein each of A₁, A₂, B₁ and B₂ is independently selected from O and S, and Z is optionally substituted alkane-di-yl or alkene-di-yl.

34. (canceled)

35. A product obtainable from the reaction of (i) Mn(CO)₅(SO₃CF₃) with [Me₄N] [acetate] under anaerobic conditions in solvent and heating, the product having CO stretching frequencies of 2027 cm⁻¹(s) and 1930 cm ⁻¹ (vs) in DCM; or (ii) Mn(CO)₅(SO₃CF₃) with potassium acetate under anaerobic conditions in solvent and heating.
